(19) United States
(12) Patent Application Publication
(10) Pub. No.: US 2007/0066640 A1
(43) Pub. Date: Mar. 22, 2007

(54) Heteroaryl-substituted pyrrolo'2,3-b1 pyridine derivatives as crf receptor antagonists

(76) Inventors: Emiliano Castiglioni, Verona (IT); Romano Di Fabio, Verona (IT); Aldo Feriani, Verona (IT); Fabrizio Micheli, Verona (IT); Fabio Sabbatini, Verona (IT); Yves St-Denis, Verona (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/542,196

(22) PCT Filed: Jan. 14, 2004
(86) PCT No.: PCT/EP04/00409

§ 371 Date: Mar. 3, 2006

Related U.S. Application Data
(60) Provisional application No. 60/440,432, filed on Jan. 16, 2003.

(51) Int. Cl. A61K 31/4745; C07D 471/02

(52) U.S. Cl. 514/300; 546/113

(57) ABSTRACT

The present invention provides compounds of formula (I) including stereoisomers, prodrugs and pharmaceutically acceptable salts or solvates thereof, to processes for their preparation, to pharmaceutical compositions containing them and to their use in the treatment of conditions mediated by corticotropin-releasing factor (CRF).

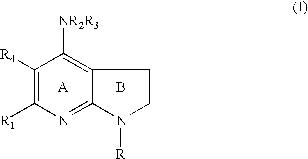

The present invention relates to bicyclic derivatives, to processes for their preparation, to pharmaceutical compositions containing them and to their use in therapy.

The first corticotropin-releasing factor (CRF) was isolated from ovine hypothalami and identified as a 41-amino acid peptide (Vale et al., Science 213: 1394-1397, 1981).

CRF has been found to produce profound alterations in endocrine, nervous and immune system function. CRF is believed to be the major physiological regulator of the basal and stress-release of adrenocorticotropic hormone (“ACTH”), Bendorphin and other proopiomelanocortin (“POMC”)-derived peptides from the anterior pituitary (Vale et al., Science 213: 1394-1397, 1981).

In addition to its role in stimulating the production of ACTH and POMC, CRF appears to be one of the pivotal central nervous system neurotransmitters and plays a crucial role in integrating the body's overall response to stress.

Administration of CRF directly to the brain elicits behavioral, physiological and endocrine responses identical to those observed for an animal exposed to a stressful environment. Accordingly, clinical data suggests that CRF receptor antagonists may represent novel antidepressant and/or anxiolytic drugs that may be useful in the treatment of the neuropsychiatric disorders manifesting hypersecretion of CRF.

The first CRF receptor antagonists were peptides (see, e.g., Rivier et al., U.S. Pat. No. 4,605,642; Rivier et al., Science 224: 889, 1984). While these peptides established that CRF receptor antagonists can attenuate the pharmacological responses to CRF, peptide CRF receptor antagonists suffer from the usual drawbacks of peptide therapeutics including lack of stability and limited oral activity. More recently, small molecule CRF receptor antagonists have been reported.

WO 95/10506 describes inter alia compounds of general formula (A) with general CRF antagonist activity 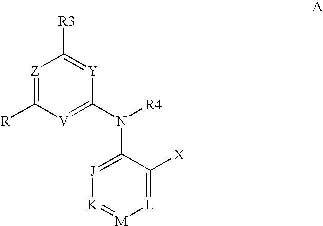
wherein Y may be CR₂₉; V may be nitrogen, Z may be carbon, R₃ may correspond to an amine derivative and R4 may be taken together with R₂₉ to form a 5-membered ring and is —CH(R₂₈) when R₂₉ is —CH(R₃₀). There are no specific disclosures of compounds corresponding to this definition.

WO 95/33750 (and in an analogously way WO01/53263 and EP773023) describes compounds of general formula (B) having CRF antagonistic activity, 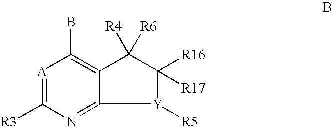
in which A and Y may be nitrogen and carbon and B may correspond to an amine derivative. There are no specific disclosures of compounds corresponding to this definition.

The present invention constitutes a novel selection of the invention object of an International patent application filed by the same Applicant WO 03/008412, describing compounds of the following general formula C: 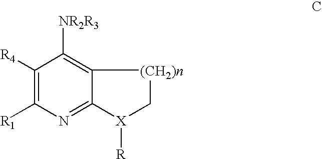
and including in the specification the preparation of the following compounds:
1-(2,4-bis-trifluoromethyl-phenyl)-6-methyl-4-(3-thiazol-2-yl-pyrazol-1-yl)-2,3-dihydro-1H-pyrrolo[2,3-b]pyridine;
3-methyl-4-[6-methyl-4-(3-thiazol-2-yl-pyrazol-1-yl)-2,3-dihydro-1H-pyrrolo[2,3-b]pyridin-1-yl]-benzonitrile;
4-[6-methyl-4-(3-thiazol-2-yl-pyrazol-1-yl)-2,3-dihydro-1H-pyrrolo[2,3-b]pyridin-1-yl]-3-trifluoromethyl-benzonitrile;
6-methyl-1-(2-methyl-4-trifluoromethoxy-phenyl)-4-(3-thiazol-2-yl-pyrazol-1-yl)-2,3-dihydro-1H-pyrrolo[2,3-b]pyridine;
1-(4-methoxy-2-methyl-phenyl)-6-methyl-4-(3-thiazol-2-yl-pyrazol-1-yl)-2,3-dihydro-1H-pyrrolo[2,3-b]pyridine
1-(2,4-bis-trifluoromethyl-phenyl)-6-methyl-4-(3-pyridin-2-y-pyrazol-1-yl)-2,3-dihydro-1H-pyrrolo[2,3-b]pyridine
4-[1,3]bipyrazolyl-1′-yl-1-(2,4-bis-trifluoromethyl-phenyl)-6-methyl-2,3-dihydro-1H-pyrrolo[2,3-b]pyridine.

Due to the physiological significance of CRF, the development of biologically-active small molecules having significant CRF receptor binding activity and which are capable of antagonizing the CRF receptor remains a desirable goal. Such CRF receptor antagonists would be useful in the treatment of endocrine, psychiatric and neurologic conditions or illnesses, including stress-related disorders in general.

While significant strides have been made toward achieving CRF regulation through administration of CRF receptor antagonists, there remains a need in the art for effective small molecule CRF receptor antagonists. There is also a need for pharmaceutical compositions containing such CRF receptor antagonists, as well as methods relating to the use thereof to treat, for example, stress-related disorders. The present invention fulfills these needs, and provides other related advantages.

In particular the invention relates to novel compounds which are potent and specific antagonists of corticotropin-releasing factor (CRF) receptors.

The present invention provides compounds of formula (I) including stereoisomers, prodrugs and pharmaceutically acceptable salts or solvates thereof 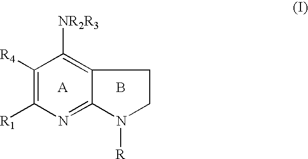
wherein
R is aryl or heteroaryl, each of which may be substituted by 1 or more Z groups;
R₁ is hydrogen, C3-C7 cycloalkyl, C1-C6 alkyl, C1-C6 alkoxy, C1-C6 thioalkyl, C2-C6 alkenyl, C2-C6 alkynyl, halo C1-C6 alkyl, halo C1-C6 alkoxy, halogen, NR₆R₇ or cyano;
R₂ and R₃ together with N form a 5-6 membered aromatic heterocycle, which is substituted by at least one group R₆ and may be further substituted by 1 to 3 R₉ groups;
R₄ hydrogen, C1-C6 alkyl, halogen or halo C1-C6 alkyl;
R₅ is a C1-C4 alkyl, —OR₆ or —NR₆R₇;
R₆ is hydrogen or C1-C6 alkyl;
R₇ is hydrogen or C1-C6 alkyl;
R₈ is a 5-6 membered aromatic heterocycle, which may be substituted by 1 to 4 R₁₀ groups;
R₉ is hydrogen, C3-C7 cycloalkyl, C3-C7 cycloalkenyl, C1-C6 alkyl, C1-C6 alkoxy, halo C1-C6 alkyl, halo C1-C6 alkoxy, hydroxy, halogen, nitro, cyano, —C(O)R₅, C(O)NR₆R₇, phenyl which may be substituted by 1 to 4 R₁₀ groups;
R₁₀ is C1-C6 alkyl, halo C1-C2 alkyl, halogen, nitro, hydroxy, halo C1-C6 alkoxy, C1-C6 alkoxy, or cyano;
Z is selected in a group consisting from halogen, C1-C6 alkyl, C1-C6 alkoxy, halo C1-C6 alkyl, C2-C6 alkenyl, C2-C6 alkynyl, halo C1-C6 alkoxy, —C(O)R₅, —NR₆R₇, nitro, cyano, and a group R₈;
and when R₄ is hydrogen, R₂ and R₃ together with N form a pyrazolyl group substituted with at least one R₈ corresponding to a thiazolyl group, and R corresponds to a phenyl group, and the substituent Z is at least one nitro group,
then in the compounds of formula (I) the nitro group is not present in the ortho position with respect to the nitrogen atom present in the 5-membered ring, named as B;
and the compounds of formula (I) don't include the following:
1-(2,4-bis-trifluoromethyl-phenyl)-6-methyl-4-(3-thiazol-2-yl-pyrazol-1-yl)-2,3-dihydro-1H-pyrrolo[2,3-b]pyridine;
3-methyl-4-[6-methyl-4-(3-thiazol-2-yl-pyrazol-1-yl)-2,3-dihydro-1H-pyrrolo[2,3-b]pyridin-1-yl]-benzonitrile;
4-[6-methyl-4-(3-thiazol-2-yl-pyrazol-1-yl)-2,3-dihydro-1H-pyrrolo[2,3-b]pyridin-1-yl]-3-trifluoromethyl-benzonitrile;
6-methyl-1-(2-methyl-4-trifluoromethoxy-phenyl)-4-(3-thiazol-2-yl-pyrazol-1-yl)-2,3-dihydro-1H-pyrrolo[2,3-b]pyridine;
1-(4-methoxy-2-methyl-phenyl)-6-methyl-4-(3-thiazol-2-yl-pyrazol-1-yl)-2,3-dihydro-1H-pyrrolo[2,3-b]pyridine
1-(2,4-bis-trifluoromethyl-phenyl)-6-methyl-4-(3-pyridin-2-yl-pyrazol-1-yl)-2,3-dihydro-1H-pyrrolo[2,3-b]pyridine
4-[1,3′]bipyrazolyl-1′-yl-1-(2,4-bis-trifluoromethyl-phenyl)-6-methyl-2,3-dihydro-1H-pyrrolo[2,3b]pyridine.

The compounds of the present invention may be in the form of and/or may be administered as a pharmaceutically acceptable salt. For a review on suitable salts see Berge et al, J. Pharm. Sci., 1977, 66, 1-19.

Typically, a pharmaceutical acceptable salt may be readily prepared by using a desired acid or base as appropriate. The salt may precipitate from solution and be collected by filtration or may be recovered by evaporation of the solvent.

Suitable addition salts are formed from adds which form non-toxic salts and examples are hydrochloride, hydrobromide, hydroiodide, sulphate, bisulphate, nitrate, phosphate, hydrogen phosphate, acetate, maleate, malate, fumarate, lactate, tartrate, citrate, formate, gluconate, succinate, piruvate, oxalate, oxaloacetate, trifluoroacetate, saccharate, benzoate, methansulphonate, ethanesulphonate, benzenesulphonate, p-toluensulphonate, methanesulphonic, ethanesulphonic, p-toluenesulphonic, and isethionate.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

In addition, prodrugs are also included within the context of this invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of structure (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

With regard to stereoisomers, the compounds of structure (I) may have one or more asymmetric carbon atom and may occur as recemates, racemic mixtures and as individual enantiomers or diastereomers. All such isomeric forms are included within the present invention, including mixtures thereof.

Where a compound of the invention contains an alkenyl or alkenylene group, cis (E) and trans (Z) isomerism may also occur. The present invention includes the individual stereoisomers of the compound of the invention and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent may also be prepared from a corresponding optically pure intermediate or by resolution, such as H.P.L.C. of the corresponding racemate using a suitable chiral support or by fractional crystallisation of the diastereoisomeric salts formed by reaction of the corresponding racemate with a suitable optically active acid or base, as appropriate.

Furthermore, some of the crystalline forms of the compounds of structure (I) may exist as polymorphs, which are included in the present invention.

The term C1-C6 alkyl as used herein as a group or a part of the group refers to a linear or branched alkyl group containing from 1 to 6 carbon atoms; examples of such groups include methyl, ethyl, propyl, isopropyl, n-butyl, isobutyl, tert butyl, pentyl or hexyl.

The term C3-C7 cycloalkyl group means a saturated hydrocarbon ring of 3 to 7 carbon atom such as, for example, cyclopropyl, cyclobutyl, cyclopentyl, cyclohexyl or cycloheptyl; while unsaturated cycloalkyls include cyclopentenyl and cyclohexenyl, and the like.

The term C3-C7 cycloalkenyl group means a non aromatic monocyclic hydrocarbon ring of 3 to 7 carbon atom, containing from one to two unsaturation such as, cyclopentenyl and cyclohexenyl, and the like.

The term halogen refers to a fluorine, chlorine, bromine or iodine atom. The term halo C1-C6 alkyl, or halo C1-C2 alkyl means an alkyl group having one or more carbon atoms and wherein at least one hydrogen atom is replaced with halogen such as for example a trifluoromethyl group and the like.

The term C2-C6 alkenyl defines straight or branched chain hydrocarbon radicals containing one or more double bond and having from 2 to 6 carbon atoms such as, for example, ethenyl, 2-propenyl, 3-butenyl, 2-butenyl, 2-pentenyl, 3-pentenyl, 3-methyl-2-butenyl or 3-hexenyl and the like.

The term C1-C6 alkoxy group may be a linear or a branched chain alkoxy group, for example methoxy, ethoxy, propoxy, prop-2-oxy, butoxy, but-2-oxy or methylprop-2-oxy and the like.

The term C1-C6 thioalkyl may be a linear or a branched chain thioalkyl group, for example thiomethyl, thioethyl, thiopropyl, thioisopropyl, thiobutyl, thiosec-butyl, thiotert-butyl and the like.

The term halo C1-C6 alkoxy group may be a C1-C6 alkoxy group as defined before substituted with at least one halogen, preferably fluorine, such as OCHF₂, or OCF₃.

The term C2-C6 alkynyl defines straight or branched chain hydrocarbon radicals containing one or more triple bond and having from 2 to 6 carbon atoms including acetylenyl, propynyl, 1-butynyl, 1-pentynyl, 3-methyl-1-butynyl and the like.

The term aryl means an aromatic carbocyclic moiety such as phenyl, biphenyl or naphthyl.

The term heteroaryl means an aromatic heterocycle ring of 5 to 10 members and having at least one heteroatom selected from nitrogen, oxygen and sulfur, and containing at least 1 carbon atom, including both mono-and bicyclic ring systems.

Representative heteroaryls include (but are not limited to) furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, benzopyrazolyl, and quinazolinyl.

The term 5-6 membered aromatic heterocycle means, a 5-6 monocyclic heterocyclic ring which is aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized. Heterocycles include some of the heteroaryls as defined above. The heterocycle may be attached via any heteroatom or carbon atom. Thus, the term include (but are not limited to) furyl, thiophenyl, pyrrolyl, tetrazolyl, pyrimidinyl, pyridinyl, pyrazinyl, pyrazolyl, thiazolyl, triazolyl, imidazolyl, oxadiazolyl, oxazolyl, and the like. Since such 5-6 membered aromatic heterocycles may be substituted by hydroxy groups, the possible tautomeric forms are also part of the present invention.

Representative compounds of this invention include compounds of general formula (II), corresponding to compounds of formula (I) in which R₄ is hydrogen and R, R₁, R₂ and R₃ are defined as above. 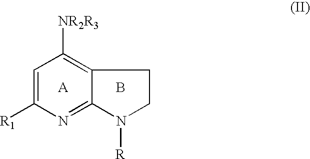
and when R₄ is hydrogen, R₂ and R₃ together with N form a pyrazolyl group substituted with at least one R₈ corresponding to a thiazolyl group, and R corresponds to a phenyl group, and the substituent Z is at least one nitro group,
then in the compounds of formula (II) the nitro group is not present in the ortho position with respect to the nitrogen atom present in the 5-membered ring, named as B;
   and the compounds of formula (II) don't include the following:
1-(2,4-bis-trifluoromethyl-phenyl)-6-methyl-4-(3-thiazol-2-yl-pyrazol-1-yl)-2,3-dihydro-1H-pyrrolo[2,3-b]pyridine;
3-methyl-4-[6-methyl-4-(3-thiazol-2-yl-pyrazol-1-yl)-2,3-dihydro-1H-pyrrolo[2,3-b]pyridin-1-yl]-benzonitrile;
4-[6-methyl-4-(3-thiazol-2-yl-pyrazol-1-yl)-2,3-dihydro-1H-pyrrolo[2,3-b]pyridin-1-yl]-3-trifluoromethyl-benzonitrile;
6-methyl-1-(2-methyl-4-trifluoromethoxy-phenyl)-4-(3-thiazol-2-yl-pyrazol-1-yl)-2,3-dihydro-1H-pyrrolo[2,3-b]pyridine;
1-(4-methoxy-2-methyl-phenyl)-6-methyl-4-(3-thiazol-2-yl-pyrazol-1-yl)-2,3-dihydro-1H-pyrrolo[2,3-b]pyridine
1-(2,4-bis-trifluoromethyl-phenyl)-6-methyl-4-(3-pyridin-2-yl-pyrazol-1-yl)-2,3-dihydro-1H-pyrrolo[2,3-b]pyridine
4-[1,3′]bipyrazolyl-1′-yl-1-(2,4-bis-trifluoromethyl-phenyl)-6-methyl-2,3-dihydro-1H-pyrrolo[2,3-b]pyridine.

In particular compounds of formula (III) are preferred, corresponding to the compounds of formula (II) 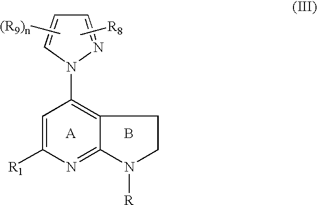
in which
R₂ and R₃ together with N form a pyrazolyl group;
n is an integer from 1 to 2;
   and R, R₁, R₆ and R₉ have the meanings as defined before;
and when R₄ is hydrogen, R₂ and R₃ together with N form a pyrazolyl group substituted with at least one R₈ corresponding to a thiazolyl group, and R corresponds to a phenyl group, and the substituent Z is at least one nitro group,
then in the compounds of formula (III) the nitro group is not present in the ortho position with respect to the nitrogen atom present in the 5-membered ring, named as B;
   and the compounds of formula (III) don't include the following:
1-(2,4-bis-trifluoromethyl-phenyl)-6-methyl-4-(3-thiazol-2-yl-pyrazol-1-yl)-2,3-dihydro-1H-pyrrolo[2,3-b]pyridine;
3-methyl-4-[6-methyl-4-(3-thiazol-2-yl-pyrazol-1-yl)-2,3-dihydro-1H-pyrrolo[2,3-b]pyridin-1-yl]-benzonitrile;
4-[6-methyl-4-(3-thiazol-2-yl-pyrazol-1-yl)-2,3-dihydro-1H-pyrrolo[2,3-b]pyridin-1-yl]-3-trifluoromethyl-benzonitrile;
6-methyl-1-(2-methyl-4-trifluoromethoxy-phenyl)-4-(3-thiazol-2-yl-pyrazol-1-yl)-2,3-dihydro-1H-pyrrolo[2,3-b]pyridine;
1-(4-methoxy-2-methyl-phenyl)-6-methyl-4-(3-thiazol-2-yl-pyrazol-1-yl)-2,3-dihydro-1H-pyrrolo[2,3-b]pyridine
1-(2,4-bis-trifluoromethyl-phenyl)-6-methyl-4-(3-pyridin-2-yl-pyrazol-1-yl)-2,3-dihydro-1H-pyrrolo[2,3-b]pyridine
4-[1,3]bipyrazolyl-1′-yl-1-(2,4-bis-trifluoromethyl-phenyl)-6-methyl-2,3-dihydro-1H-pyrrolo[2,3-b]pyridine.

Preferred are the compounds of formula (IV), corresponding to compounds of formula (III), 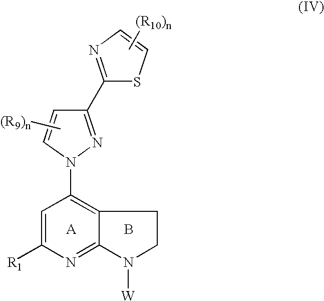
in which:
R₈ is a thiazolyl derivative;
n is an integer from 1 to 2;
R corresponds to W and
W is a pyridine derivative, which may be substituted by 1 to 4 Z groups, as defined above; and
R₁, R₉ and R₁₀ are defined as above.

Particularly preferred are the compounds of formula (IVa), 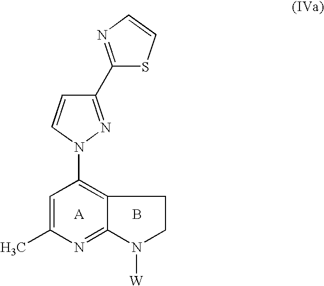
in which W is defined as above.

Specific examples of compounds of general formula (IVa) are included in the Experimental Part.

Equally preferred are compounds of formula (V), corresponding to compounds of formula (III), 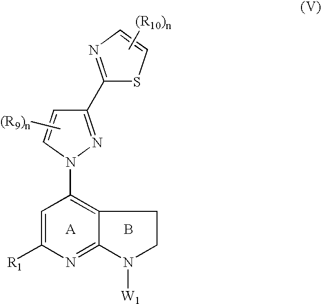
in which:
R₈ is a thiazolyl derivative;
n is an integer from 1 to 2;
R corresponds to W₁ and
W₁ is a phenyl derivative substituted by 2 to 4 Z₁ groups;
Z₁ is selected in a group consisting from halogen, C1-C6 alkyl, C1-C6 alkoxy, halo C1-C6 alkyl, C2-C6 alkenyl, C2-C6 alkynyl, halo C1-C6 alkoxy, —C(O)R₅, —NR₆R₇, cyano, and a group R₈;
R₁, R₉ and R₁₀ are defined as above;
   and the compounds of formula (V) don't include the following:
1-(2,4-bis-trifluoromethyl-phenyl)-6-methyl-4-(3-thiazol-2-yl-pyrazol-1-yl)-2,3-dihydro-1H-pyrrolo[2,3-b]pyridine;
3-methyl-4-[6-methyl-4-(3-thiazol-2-yl-pyrazol-1-yl)-2,3-dihydro-1H-pyrrolo[2,3-b]pyridin-1-yl]-benzonitrile;
4-[6-methyl-4-(3-thiazol-2-yl-pyrazol-1-yl)-2,3-dihydro-1H-pyrrolo[2,3-b]pyridin-1-yl]-3-trifluoromethyl-benzonitrile;
6-methyl-1-(2-methyl-4-trifluoromethoxy-phenyl)-4-(3-thiazol-2-yl-pyrazol-1-yl)-2,3-dihydro-1H-pyrrolo[2,3-b]pyridine;
1-(4-methoxy-2-methyl-phenyl)-6-methyl-4-(3-thiazol-2-yl-pyrazol-1-yl)-2,3-dihydro-1H-pyrrolo[2,3-b]pyridine
1-(2,4-bis-trifluoromethyl-phenyl)-6-methyl-4-(3-pyridin-2-yl-pyrazol-1-yl)-2,3-dihydro-1H-pyrrolo[2,3-b]pyridine
4-[1,3]bipyrazolyl-1′-yl-1-(2,4-bis-trifluoromethyl-phenyl)-6-methyl-2,3-dihydro-1H-pyrrolo[2,3-b]pyridine.

Particularly preferred are the compounds of formula (Va), 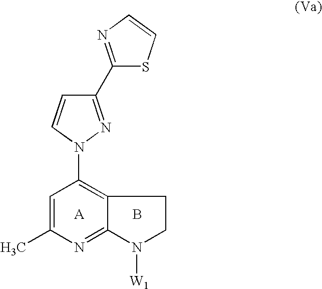
in which W₁ is defined as above.

Specific examples of compounds of general formula (Va) are included in the Experimental Part.

Equally preferred are compounds of formula (VI), corresponding to compounds of formula (III). 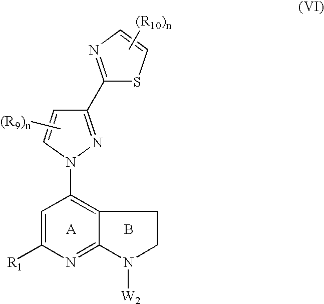
in which:
R₈ is a thiazolyl derivative;
n is an integer from 1 to 2;
R corresponds to W₂ and
W₂ is a phenyl derivative, which may be substituted by 2 to 4 Z groups as defined above provided that at least one Z group is nitro and further provided that the nitro group is not in the ortho position with respect to the nitrogen atom of the 5-membered ring named as B; and
R₁, R₉ and R₁₀ are defined as above.

Even more preferred are the compounds of formula (V), in which R is a phenyl derivative containing a nitro group in para position respect to the nitrogen atom of the 5-membered ring B.

Particularly preferred are the compounds of formula (VIa), 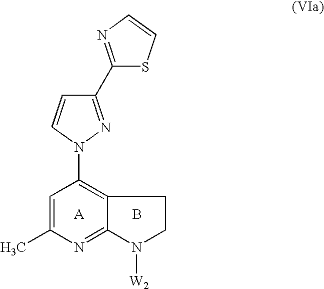
in which W₂ is defined as above.

Specific examples of compounds of general formula (VIa) are included in the Experimental Part.

Equally preferred are compounds of formula (VII), 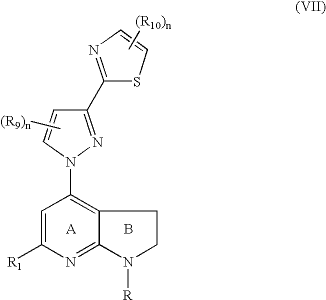
in which
R₈ is a pyrazolyl derivative;
n is an integer from 1 to 2;
R, R₉ and R₁₀ are defined as above.

Particularly preferred are the compounds of formula (VIIa), 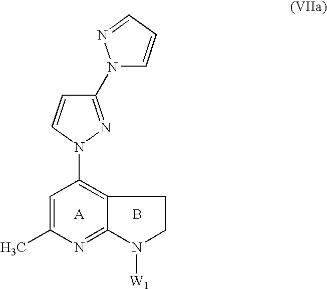
in which W₁ is defined as above.

Specific examples of compounds of general formula (VIIa) are included in the Experimental Part.

Equally preferred are the compounds of formula (VIII), 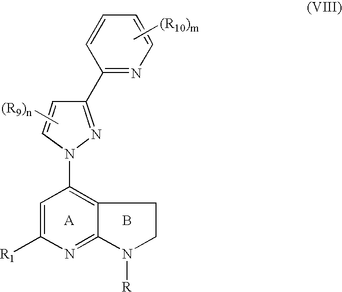
in which
R₈ is a pyridine derivative;
n is an integer from 1 to 2;
m is an integer from 1 to 3;
R, R₉ and R₁₀ are defined as above.

Particularly preferred are the compounds of formula (VIIa), corresponding to the compounds of formula (VIII) 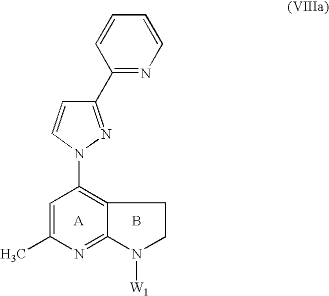
in which W₁ is defined as above.

Specific examples of compounds of general formula (VIIIa) are included in the Experimental Part.

Preferred compounds according to the invention are:
6-methyl-1-[6-(methyloxy)-2-(trifluoromethyl)-3-pyridinyl]-4-[3-(1,3-thiazol-2-yl)-1H-pyrazol-1-yl]-2,3-dihydro-1H-pyrrolo[2,3-b]pyridine;
1-[2,6-bis(methyloxy)-3-pyridinyl]-6-methyl-4-[3-(1,3-thiazol-2-yl)-1H-pyrazol-1-yl]-2,3-dihydro-1H-pyrrolo[2,3-b]pyridine;
N,N,4-trimethyl-5-{6-methyl-4-[3-(1,3-thiazol-2-yl)-1H-pyrazol-1-yl]-2,3-dihydro-1H-pyrrolo[2,3-b]pyridin-1-yl}-2-pyridinamine;
1-(2-difluoromethyl-4-methoxy-phenyl)-6-methyl-4-(3-thiazol-2-yl-pyrazol-1-yl)-2,3-dihydro-1H-pyrrolo[2,3-b]pyridine;
1-(2-chloro-4-methoxy-phenyl)-6-methyl-4-(3-thiazol-2-yl-pyrazol-1-yl)-2,3-dihydro-1H-pyrrolo[2,3-b]pyridine;
1-[2,4-bis(methyloxy)phenyl]-6-methyl-4-[3-(1,3-thiazol-2-yl)-1H-pyrazol-1-yl]-2,3-dihydro-1H-pyrrolo[2,3-b]pyridine;
3-chloro-4-{6-methyl-4-[3-(1,3-thiazol-2-yl)-1H-pyrazol-1-yl]-2,3-dihydro-1H-pyrrolo[2,3-b]pyridin-1-yl}benzonitrile;
4-{6-methyl-4-[3-(1,3-thiazol-2-yl)-1H-pyrazol-1-yl]-2,3-dihydro-1H-pyrrolo[2,3-b]pyridin-1-yl}-3-[(trifluoromethyl)oxy]benzonitrile;
3-ethyl-4-{6-methyl-4-[3-(1,3-thiazol-2-yl)-1H-pyrazol-1-yl]-2,3-dihydro-1H-pyrrolo[2,3-b]pyridin-1-yl}benzonitrile;
1-(4-fluoro-2-methylphenyl)-6-methyl-4-[3-(1,3-thiazol-2-yl)-1H-pyrazol-1-yl]-2,3-dihydro-1H-pyrrolo[2,3-b]pyridine;
6-methyl-1-[2-methyl-4-(1H-pyrazol-1-yl)phenyl]-4-[3-(1,3-thiazol-2-yl)-1H-pyrazol-1-yl]-2,3-dihydro-1H-pyrrolo[2,3-b]pyridine;
6-methyl-1-[4-nitro-2-(trifluoromethyl)phenyl]-4-[3-(1,3-thiazol-2-yl)-1H-pyrazol-1-yl]-2,3-dihydro-1H-pyrrolo[2,3-b]pyridine;
4-[4-(1′H-1,3′-bipyrazol-1′-yl)-6-methyl-2,3-dihydro-1H-pyrrolo[2,3-b]pyridin-1-yl]-3-methylbenzonitrile;
4-[4-(1′H-1,3′-bipyrazol-1′-yl)-6-methyl-2,3-dihydro-1H-pyrrolo[2,3-b]pyridin-1-yl]-3-(trifluoromethyl)benzonitrile;
4-[4-(1′H-1,3′-bipyrazol-1′-yl)-6-methyl-2,3-dihydro-1H-pyrrolo[2,3-b]pyridin-1-yl]-3-chlorobenzonitrile;
1′-{6-methyl-1-[2-methyl-(methyloxy)phenyl]-2,3-dihydro-1H-pyrrolo[2,3-b]pyridin-4-yl}-1′H-1,3′-bipyrazole;
3-methyl-4-{6-methyl-4-[3-(2-pyridinyl)-1H-pyrazol-1-yl]-2,3-dihydro-1H-pyrrolo[2,3-b]pyridin-1-yl}benzonitrile;
3-chloro-4-{6-methyl-4-[3-(2-pyridinyl)-1H-pyrazol-1-yl]-2,3-dihydro-1H-pyrrolo[2,3-b]pyridin-1-yl}benzonitrile;
4-{6-methyl-4-[3-(2-pyridinyl)-1H-pyrazol-1-yl]-2,3-dihydro-1H-pyrrolo[2,3-b]pyridin-1-yl}-3-(trifluoromethyl)benzonitrile.

In general, the compounds of structure (I) may be made according to the organic synthesis techniques known to those skilled in this field, as well as by the representative methods set forth in the Examples.

Compounds of formula (I), and salts and solvates thereof, may be prepared by the general methods outlined hereinafter. In the following description, the groups R, R₁, R₂, R₃, R₄, R₅, R₆, R₇, R₈, R₉, R₁₀, Z, W, W₁, W₂, m and n have the meanings as previously defined for compounds of formula (I) unless otherwise stated.

Compounds of formula (I) may be conveniently prepared, starting from compounds of formula (IIIB), according to the following Scheme 1: 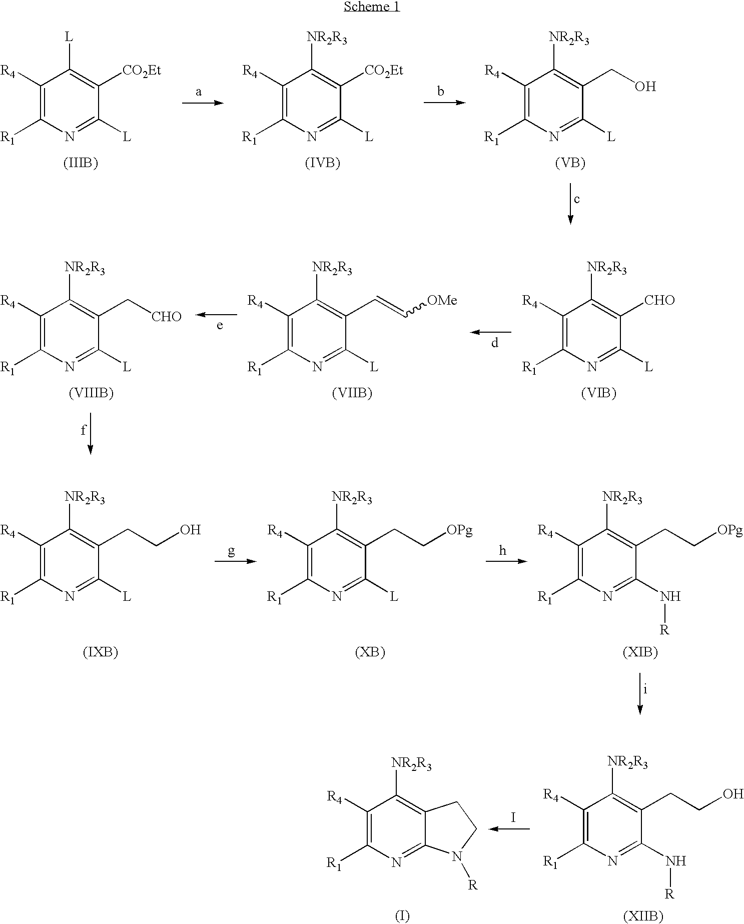
in which
step a stands for conversion of the leaving group L, selected in a group consisting from: halogen or reactive residue of sulphonic acid (e.g. mesylate, tosylate), preferably chloride, in the amino group of compounds (IVB), by reaction with the suitable amine NR₂R₃ in basic conditions;
step b stands for reduction of the ester group with a suitable reducing agent (such as DIBAI-H) to hydroxy group of compounds (VB);
step c stands for oxidation of the hydroxy group with a suitable oxidising agent (such as Dess-Martin periodinane) to aldehyde group of compound (VIB);
step d stands for formation of the aldehyde group of compounds (VIIIB) by Wittig reaction in the usual conditions, through formation of enol ether followed by acid hydrolysis (step e);
step f stands for reduction of the aldehyde group with a suitable reducing agent (such as NaBH₄) to hydroxy group of compounds (IXB);
step g stands for conversion of the hydroxy group in the suitably protected hydroxy group of compounds (XB)(such as TBS: tert-butyldimethylsilyl);
step h stands for Buchwald reaction by coupling with the suitable amine RNH₂;
step i stands for deprotection reaction to give the hydroxy group of compounds (XIIB);
step l stands for intramolecular cyclisation after conversion of the hydroxy group of compounds (XIIB) in a suitable leaving group (such as bromide, by reaction with CBr₄ and PPh₃) to give the final compounds (I).

Alternatively, compounds of formula (I) may be conveniently prepared according to the following Scheme 2: 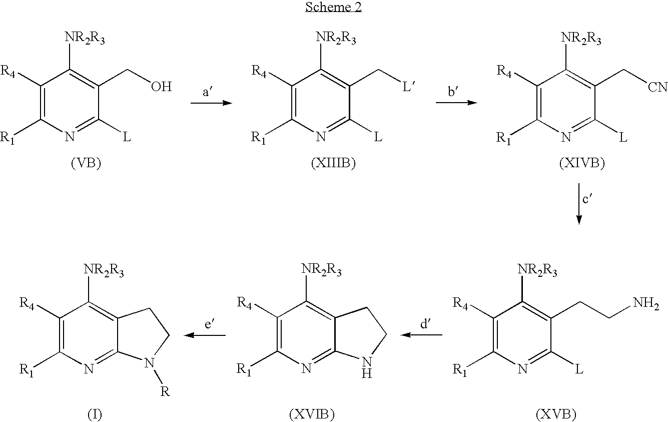
in which
step a′ stands for conversion of the hydroxy group in a suitable leaving group L′ of compounds (XIIIB), which, independently from L, has the same definition (e.g mesylate, by reaction with MsCl and Et₃N);
step b′ stands for conversion of L′ in the cyano derivative of compounds (XIVB) by reaction with, e.g. KCN in an aprotic dipolar solvent, like DMF;
step c′ stands for reduction of the cyano group with a suitable reducing agent agent (such as BH₃-THF) to the amino group of compound (XVB);
step d′ stands for intramolecular cyclisation of compounds (XVB) by heating in a suitable solvent (such as NMP) at high temperature or intramolecular Copper catalysed cyclisation;
step e′ see step h above. The reaction is done with a suitable aryl halide to give the final compounds (I).

Alternatively, compounds of formula (I) may be conveniently prepared according to the following Scheme 3: 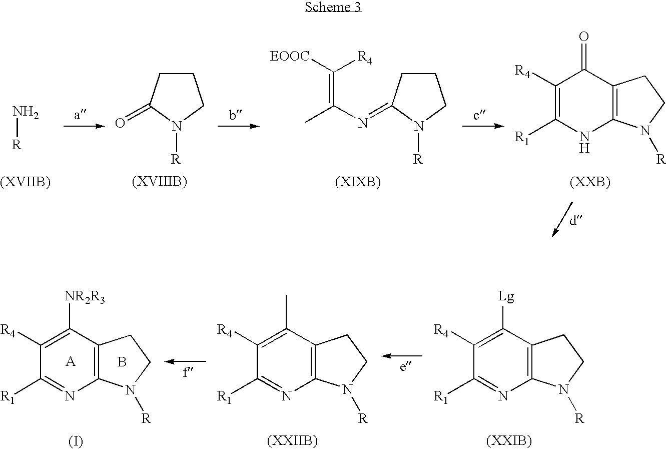
in which:
step a″ stands for the formation of the pyrrolidinone moiety of compounds (XVIIIB), which will form the cycle B present in the final compounds (I), by reacting the compounds (XVIIB) with a reactive derivative of the butyric acid, such as 4-chlorobutyryl chloride; followed by a cyclisation reaction in basic conditions (e.g. KOtBu);
step b″ stands for amidine formation by reacting the compounds (XIXB) with 3-aminocrotonate and POCl₃;
step c″ stands for the cyclisation of the compounds (XIXB) in basic conditions (e.g. NaH) to give the pyridinone precursor of cycle A in the final compounds (I);
step d″ stands for the formation of a reactive derivative (i.e. a leaving group, Lg) of the pyridinone (for example selected in a group consisting by triflate, halogen, mesylate) of compounds (XXIB) by reaction with, for example, triflic anhydride;
step e″ stands for nucleophilic displacement of the leaving group of compounds (XXIB) to give the iodinated compounds (XXIIB);
step f″ stands for the arylation reaction with the suitable pyrazole derivative by a metal catalysed coupling reaction (for example a Buchwald reaction) procedure to give the final compounds (I).

Another aspect of the present invention is to furnish a convenient process for preparing derivatives in which the group R contains —CHF₂ as one or more of the groups Z.

Such process comprises the following steps described in Scheme 4. The process has been exemplified for R corresponding to phenyl derivative, but it is not limiting its appropriateness to the various meanings of R, as defined above. 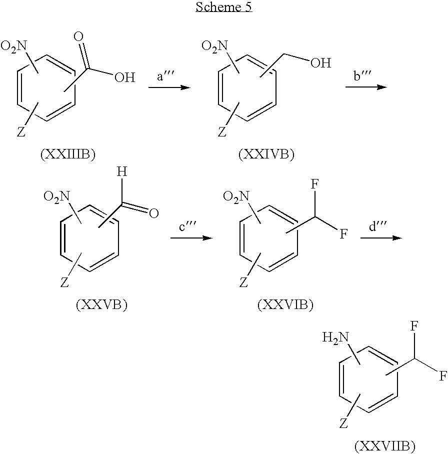
step a′″ stands for the reduction of the acid group present in (XXIIIB) to the hydroxy group of compounds (XXIVB) by a suitable reducing agent (such as treatment with cyanuric chloride and NMM and then reduction with NaBH₄);
step b′″ stands for the oxidation to the aldehyde group of compounds (XXVB) by a suitable oxidising agent (such as Dess Martin periodinane);
step c′″ stands for the fluorination of the aldehyde group by a suitable fluorinating agent (such as DAST: (diethylamino)sulphurtrifluoride);
step d′″ stands for the final reduction of the nitro group to the amino group of compounds (XXVIIB) by a suitable reducing agent.

Compounds of formula (IIIB), (VB), (XVIIB) and (XXIIIB) are known compounds or may be prepared according to known methods in the literature.

Compounds of formula (III), (IIIa), (IV), (IVa), (V), (Va), (VI), (VIa), (VII), (VIIa), (VIII), and (VIIIa) may be prepared according to the previous Schemes 1, 2 and 3, once prepared the heterocyclic reactive residue according to known methods to the skilled in the art.

Those skilled in the art will appreciate that in the preparation of the compound of the invention or a solvate thereof it may be necessary and/or desirable to protect one or more sensitive groups in the molecule to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl type protecting groups (e.g. benzyl, trityl, chlorotrityl). Examples of suitable oxygen protecting groups may include for example alky silyl groups, such as trimethylsilyl or tert-butyldimethylsilyl; alkyl ethers such as tetrahydropyranyl or tert-butyl; or esters such as acetate

Pharmaceutical acceptable salts may also be prepared from other salts, including other pharmaceutically acceptable salts, of the compound of formula (I) using conventional methods.

The compounds of formula (I) may readily be isolated in association with solvent molecules by crystallisation or evaporation of an appropriate solvent to give the corresponding solvates.

When a specific enantiomer of a compound of general formula (I) is required, this may be obtained for example by resolution of a corresponding enantiomeric mixture of a compound of formula (I) using conventional methods. Thus the required enantiomer may be obtained from the racemic compound of formula (I) by use of chiral HPLC procedure.

The subject invention also includes isotopically-labelled compounds, which are identical to those recited in formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁸Cl, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labelled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I and following of this invention can generally be prepared by carrying out the procedures disclosed in the Schemes and/or in the Examples below, by substituting a readily available isotopically labelled reagent for a non-isotopically labelled reagent.

The CRF receptor antagonists of the present invention demonstrate activity at the CRF receptor site including CRF 1 and CRF 2 receptors and may be used in the treatment of conditions mediated by CRF or CRF receptors.

The effectiveness of a compound as a CRF receptor antagonist may be determined by various assay methods. Suitable CRF antagonists of this invention are capable of inhibiting the specific binding of CRF to its receptor and antagonizing activities associated with CRF. A compound of structure (I) may be assessed for activity as a CRF antagonist by one or more generally accepted assays for this purpose, including (but not limited to) the assays disclosed by DeSouza et al. (J. Neuroscience 7: 88, 1987) and Battaglia et al. (Synapse 1: 572, 1987).

The CRF receptors-binding assay was performed by using the homogeneous technique of scintillation proximity (SPA). The ligand binds to recombinant membrane preparation expressing the CRF receptors which in turn bind to wheatgerm agglutinin coated SPA beads. In the Experimental Part will be disclosed the details of the experiments.

With reference to CRF receptor binding affinities, CRF receptor antagonists of this invention have a Ki less than 10 μM. In a preferred embodiment of this invention, a CRF receptor antagonist has a Ki comprised in a range from 0.1 μM and 10 μM.

In a more preferred embodiment the value of Ki is less than 0.1 μM. As set forth in greater detail below, the Ki values of representative compounds of this invention were assayed by the methods set forth in Example 6.

Compounds of the invention are useful in the treatment of central nervous system disorders where CRF receptors are involved. In particular in the treatment or prevention of major depressive disorders including bipolar depression, unipolar depression, single or recurrent major depressive episodes with or without psychotic features, catatonic features, melancholic features, atypical features or postpartum onset, the treatment of anxiety and the treatment of panic disorders. The term anxiety includes anxiety disorders, such as panic disorders with or without agoraphobia, agoraphobia, phobias, for example, social phobias or agoraphobia, obsessive-compulsive disorder, stress disorders including post-traumatic stress disorders, generalised anxiety disorders, acute stress disorders and mixed anxiety-depression disorders. Other mood disorders encompassed within the term major depressive disorders include dysthymic disorder with early or late onset and with or without atypical features, neurotic depression, post traumatic stress disorders and social phobia; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood. Major depressive disorders may also result from a general medical condition including, but not limited to, myocardial infarction, diabetes, miscarriage or abortion, etc.

Compounds of the invention are also useful in the treatment or prevention of schizophrenic disorders including paranoid schizophrenia, disorganised schizophrenia, catatonic schizophrenia, undifferentiated schizophrenia, residual schizoprenia.

Compounds of the invention are useful as analgesics. In particular they are useful in the treatment of traumatic pain such as postoperative pain; traumatic avulsion pain such as brachial plexus; chronic pain such as arthritic pain such as occurring in osteo-, rheumatoid or psoriatic arthritis; neuropathic pain such as post-herpetic neuralgia, trigeminal neuralgia, segmental or intercostal neuralgia, fibromyalgia, causalgia, peripheral neuropathy, diabetic neuropathy, chemotherapy-induced neuropathy, AIDS related neuropathy, occipital neuralgia, geniculate neuralgia, glossopharyngeal neuralgia, reflex sympathetic dystrophy, phantom limb pain; various forms of headache such as migraine, acute or chronic tension headache, temporomandibular pain, maxillary sinus pain, cluster headache; odontalgia; cancer pain; pain of visceral origin; gastrointestinal pain; nerve entrapment pain; sport's injury pain; dysmennorrhoea; menstrual pain; meningitis; arachnoiditis; musculoskeletal pain; low back pain e.g. spinal stenosis; prolapsed disc; sciatica; angina; ankylosing spondyolitis; gout; burns; scar pain; itch; and thalamic pain such as post stroke thalamic pain.

Compounds of the invention are also useful for the treatment of dysfunction of appetite and food intake and in circumstances such as anorexia, anorexia nervosa and bulimia.

Compounds of the invention are also useful in the treatment of sleep disorders including dysomnia, insomnia, sleep apnea, narcolepsy, and circadian ritmic disorders.

Compounds of the invention are also useful in the treatment or prevention of cognitive disorders. Cognitive disorders include dementia, amnestic disorders and cognitive disorders not otherwise specified.

Furthermore compounds of the invention are also useful as memory and/or cognition enhancers in healthy humans with no cognitive and/or memory deficit.

Compounds of the invention are also useful in the treatment of tolerance to and dependence on a number of substances. For example, they are useful in the treatment of dependence on nicotine, alcohol, caffeine, phencyclidine (phencyclidine like compounds), or in the treatment of tolerance to and dependence on opiates (e.g. cannabis, heroin, morphine) or benzodiazepines; in the treatment of cocaine, sedative ipnotic, amphetamine or amphetamine-related drugs (e.g. dextroamphetamine, methylamphetamine) addiction or a combination thereof.

Compounds of the invention are also useful as anti-inflammatory agents. In particular they are useful in the treatment of inflammation in asthma, influenza, chronic bronchitis and rheumatoid arthritis; in the treatment of inflammatory diseases of the gastrointestinal tract such as Crohn's disease, ulcerative colitis, postoperative gastric ileus (POI), inflammatory bowel disease (IBD) and non-steroidal anti-inflammatory drug induced damage; inflammatory diseases of the skin such as herpes and eczema; inflammatory diseases of the bladder such as cystitis and urge incontinence; and eye and dental inflammation.

Compounds of the invention are also useful in the treatment of allergic disorders, in particular allergic disorders of the skin such as urticaria, and allergic disorders of the airways such as rhinitis.

Compounds of the invention are also useful in the treatment of emesis, i.e. nausea, retching and vomiting. Emesis includes acute emesis, delayed emesis and anticipatory emesis. The compounds of the invention are useful in the treatment of emesis however induced. For example, emesis may be induced by drugs such as cancer chemotherapeutic agents such as alkylating agents, e.g. cyclophosphamide, carmustine, lomustine and chlorambucil; cytotoxic antibiotics, e.g. dactinomycin, doxorubicin, mitomycin-C and bleomycin; anti-metabolites, e.g. cytarabine, methotrexate and 5-fluorouracil; vinca alkaloids, e.g. etoposide, vinblastine and vincristine; and others such as cisplatin, dacarbazine, procarbazine and hydroxyurea; and combinations thereof; radiation sickness; radiation therapy, e.g. irradiation of the thorax or abdomen, such as in the treatment of cancer; poisons; toxins such as toxins caused by metabolic disorders or by infection, e.g. gastritis, or released during bacterial or viral gastrointestinal infection; pregnancy; vestibular disorders, such as motion sickness, vertigo, dizziness and Meniere's disease; post-operative sickness; gastrointestinal obstruction; reduced gastrointestinal motility; visceral pain, e.g. myocardial infarction or peritonitis; migraine; increased intercranial pressure; decreased intercranial pressure (e.g. altitude sickness); opioid analgesics, such as morphine; and gastro-oesophageal reflux disease, acid indigestion, over-indulgence of food or drink, acid stomach, sour stomach, waterbrash/regurgitation, heartburn, such as episodic heartburn, nocturnal heartburn, and meal-induced heartburn and dyspepsia.

Compounds of the invention are of particular use in the treatment of gastrointestinal disorders such as irritable bowel syndrome (IBS); skin disorders such as psoriasis, pruritis and sunburn; vasospastic diseases such as angina, vascular headache and Reynaud's disease; cerebral ischeamia such as cerebral vasospasm following subarachnoid haemorrhage; fibrosing and collagen diseases such as scleroderma and eosinophilic fascioliasis; disorders related to immune enhancement or suppression such as systemic lupus erythematosus and rheumatic diseases such as fibrositis; and cough.

Compounds of the invention are useful for the treatment of neurotoxic injury which follows cerebral stroke, thromboembolic stroke, hemorrhagic stroke, cerebral ischemia, cerebral vasospam, hypoglycemia, hypoxia, anoxia, perinatal asphyxia cardiac arrest.

The invention therefore provides a compound of formula (I) or a pharmaceutically acceptable salt or solvate thereof for use in therapy, in particular in human medicine.

There is also provided as a further aspect of the invention the use of a compound of formula (I) or a pharmaceutically acceptable salt or solvate thereof in the preparation of a medicament for use in the treatment of conditions mediated by CRF.

In an alternative or further aspect there is provided a method for the treatment of a mammal, including man, in particular in the treatment of condition mediated by CRF, comprising administration of an effective amount of a compound of formula (I) or a pharmaceutically acceptable salt or a solvate thereof.

While it is possible that, for use in therapy, a compound of the present invention may be administered as the raw chemical, it is preferable to present the active ingredient as a pharmaceutical formulation e.g. when the agent is in admixture with a suitable pharmaceutical excipient, diluent or carrier selected with regard to the intended route of administration and standard pharmaceutical practice.

In a further aspect, the invention provides a pharmaceutical composition comprising at least one compound of the invention or a pharmaceutically acceptable derivative thereof in association with a pharmaceutically acceptable carrier and/or excipient. The carrier and/or excipient must be “acceptable” in the sense of being compatible with the other ingredients of the formulation and not deletrious to the receipient thereof.

Accordingly, the present invention further provides a pharmaceutical formulation comprising at least one compound of the invention or a pharmaceutically acceptable derivative thereof, in association with a pharmaceutically acceptable carrier and/or excipient. The carrier and/or excipient must be “acceptable” in the sense of being compatible with the other ingredients of the formulation and not deletrious to the receipient thereof.

There is further provided by the present invention a process of preparing a pharmaceutical composition, which process comprises mixing at least one compound of the invention or a pharmaceutically acceptable derivative thereof, together with a pharmaceutically acceptable carrier and/or excipient.

The pharmaceutical compositions may be for human or animal usage in human and veterinary medicine and will typically comprise any one or more of a pharmaceutically acceptable diluent, carrier or excipient. Acceptable carriers or diluents for therapetic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the agents of the present invention may also be used in combination with a cyclodextrin. Cyclodextrins are known to form inclusion and non-inclusion complexes with drug molecules. Formation of a drugcyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta and gamma-cyclodextrins are most commonly used and suitable examples are described in WO-A-91/11172, WO-A-94/02518 and WO-A-98/55148.

In a preferred embodiment, the agents of the present invention are delivered systemically (such as orally, buccally, sublingually), more preferably orally.

Hence, preferably the agent is in a form that is suitable for oral delivery.

It is to be understood that not all of the compounds need be administered by the same route. Likewise, if the composition comprises more than one active component, then those components may be administered by different routes.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see International Patent Application No. WO 02/00196 (SmithKline Beecham).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the composition may take the form of tablets or formulated in conventional manner.

The compounds of the invention may be formulated for parenteral administration by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unitary dose device or alternatively as a powder mix with a suitable carrier for administration using a suitable delivery device.

A proposed dose of the compounds of the invention is 1 to about 1000 mg per day. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected.

Thus for parenteral administration a daily dose will typically be in the range of 1 to about 100 mg, preferably 1 to 80 mg per day. For oral administration a daily dose will typically be within the range 1 to 300 mg e.g. 1 to 100 mg.

### EXAMPLES

In the Intermediates and Examples unless otherwise stated:

Melting points (m.p.) were determined on a Gallenkamp m.p. apparatus and are uncorrected. All temperatures refers to ° C. Infrared spectra were measured on a FT-IR instrument. Proton Magnetic Resonance (¹H-NMR) spectra were recorded at 400 MHz, chemical shifts are reported in ppm downfield (d) from Me₄Si, used as internal standard, and are assigned as singlets (s), doublets (d), doublets of doublets (dd), triplets (t), quartets (q) or multiplets (m). Column chromathography was carried out over silica gel (Merck AG Darmstaadt, Germany). The following abbreviations are used in text: EtOAc=ethyl acetate, cHex=cyclohexane, CH₂Cl₂=dichloromethane, Et₂O=dietyl ether, DMF=N,N′-dimethylformamide, NMP=N-methylpyrrolidinone, DIPEA=N,N-diisopropylethylamine, DME=ethylene glycol dimethyl ether, MeOH=methanol, Et₃N=triethylamine, TFA=trifluoroacetic acid, THF=tetrahydrofuran, DIBAL-H=diisobutylaluminium hydride, DMAP=dimethylaminopyridine, LHMDS=lithium hexamethyldisilazane, DMA=dimethylacetamide, NMM=N-methylmorpholine, DAST=(diethylamino)sulfur trifluoride, Tlc refers to thin layer chromatography on silica plates, and dried refers to a solution dried over anhydrous sodium sulphate; r.t. (RT) refers to room temperature.

Intermediate 1

### Ethyl 2,4-dichloro-6-methyl-3-pyridinecarboxylate

The title compound was prepared according to an already published procedure: Mittelbach, Martin; Synthesis, 1988, 6, p. 479-80.

Intermediate 2

### Ethyl 2-chloro-6-methyl-4-[3-(1,3-thiazol-2-yl)-1H-pyrazol-1-yl]-3-pyridinecarboxylate

To a solution of 2-(1H-pyrazol-3-yl)-1,3-thiazole (7.71 g, 1.05 eq) in anh. DMF (61 mL), at 0° C., under N₂, was added NaH 60% in mineral oil (2.03 g, 1.05 eq) and the reaction mixture was stirred for 10 min. at 0° C. and then for 1 hr at room temperature. Intermediate 1 (11.34 g, 48.0 mmol) was then added as a solution in anh. DMF (35 mL) at 0° C. and the resulting solution was heated at 110° C. for 3 hr. The reaction was then quenched with water, extracted with EtOAc, washed with brine, dried over anh. Na₂SO₄, filtered and concentrated in vacuo. The crude product was purified by flash chromatography (silica gel, cHex/EtOAc 7:3) to give 7.02 g of the title compound as a white solid.

NMR (¹H, CDCl₃): δ 7.91 (d, 1H), 7.91 (d, 1H), 7.41 (d, 1H), 7.31 (s, 1H), 7.18 (d, 1H), 4.50 (q, 2H), 2.78 (s, 3H), 1.25 (t, 3H).

MS (m/z): 349 [MH]⁺.

Intermediate 3

### {2-Chloro-6-methyl-4-[3-(1,3-thiazol-2-yl)-1H-pyrazol-1-yl]-3-pyridinyl}methanol

To a solution of intermediate 2 (1.5 g, 4.3 mmol) in anh. CH₂Cl₂ (30 mL), at −78° C., under N₂, was added DIBAI-H 1.0 M in cyclohexane (12.9 mL, 3.0 eq). The reaction mixture was stirred for 1 hr at −78° C. and then for 1 hr at room temperature. The reaction was then quenched with a saturated solution of Rochelle's salt, extracted with EtOAc, washed with brine, dried over anh. Na₂SO₄, filtered and concentrated in vacuo. The crude product was purified by flash chromatography (silica gel, cHex/EtOAc 1:1) to give 1.02 g of the title compound as a white solid.

NMR (¹H, CDCl₃): δ 8.05 (d, 1H), 7.90 (d, 1H), 7.40 (d, 1H), 7.25 (s, 1H), 7.10 (d, 1H), 4.65 (S, 2H), 4.0 (bs, 1H), 2.60 (s, 3H).

MS (m/z): 307 [M]⁺.

Intermediate 4

### 2-chloro-6-methyl-4-[3-(1,3-thiazol-2-yl)-1H-pyrazol-1-yl]-3-pyridinecarbaldehyde

To a solution of intermediate 3 (150 mg, 0.5 mmol) in anh. CH₂Cl₂ (5 mL), at room temperature, under N₂, was added the Dess Martin periodinane (237 mg, 1.12 eq) and the reaction mixture was stirred for 1 hr at room temperature. The reaction was then quenched with a solution of 0.5 g of sodium thiosulfate dissolved in a saturated solution of sodium bicarbonate, extracted with EtOAc, washed with brine, dried over Na₂SO₄, filtered and concentrated in vacuo. The crude product was purified by flash chromatography (silica gel, cHex/EtOAc 1:1) to give 124 mg of the title compound as a white solid.

NMR (¹H, CDCl₃): δ 10.4 (s, 1H), 8.0-7.9 (2d, 2H), 7.40 (2d, 2H), 7.10 (s, 1H), 2.70 (s, 3H).

MS (m/z): 305 [MH]⁺.

Intermediate 5

### 2-Chloro-6-methyl-3-[(E)-2-(methyloxy)ethenyl]-4-[3-(1,3-thiazol-2-yl)-1H-pyrazol-1-yl]pyridine

To a solution of (methoxymethyl)-triphenylphosphonium chloride (4.24 g, 3 eq) in anh. THF (20 mL), at 0° C., under N₂, was added n-BuLi 1.6 M in cyclohexane (7.73 ml, 12.37 mmol) and the reaction mixture was brought to room temperature and then stirred for 15 min. A solution of intermediate 4 (1.25 g, 4.1 mmol) in anh. THF (15 mL) was then added and the reaction was stirred at room temperature for 1.5 hr. The reaction was then quenched with water, extracted with EtOAc, washed with brine, dried over anh. Na₂SO₄, filtered and concentrated in vacuo. The crude product was purified by flash chromatography (silica gel, cHex/EtOAc 4:1) to give 961 mg of the title compound as a white solid (E:Z=3:2 mixture, used as such in the next step).

NMR (¹H, CDCl₃) principal E product: δ 7.90 (m, 1H), 7.83 (m, 1H), 7.38 (m, 1H), 7.05 (m, 1H), 7.00 (m, 1H), 6.51 (d, 1H), 5.63 (d, 1H), 3.64 (s, 3H), 2.60 (s, 3H).

MS (m/z): 333 [MH]⁺.

Intermediate 6

### {2-chloro-6-methyl-4-[3-(1,3-thiazol-2-yl)-1H-pyrazol-1-yl]-3-pyridinyl}acetaldehyde

To a solution of intermediate 5 (936 mg, 2.8 mmol) in anh. THF (15 mL) was added 6N HCl (21 ml, 45 eq) and the reaction mixture was stirred at room temperature for 15 hr. The reaction was then quenched with sat. aq. NaHCO₃ until pH=7, extracted with EtOAc, washed with brine, dried over anh. Na₂SO₄, filtered and concentrated in vacuo to give 893 mg of the title compound as a white solid, which was used in the next step without further purification.

NMR (¹H, CDCl₃): δ 9.80 (s, 1H), 7.90-7.80 (2d, 2H), 7.70 (d, 1H), 7.20 (d, 1H), 7.0 (s, 1H), 4.25 (s, 2H), 2.70 (s, 3H).

MS (m/z): 319 [MH]⁺.

Intermediate 7

### 2-{2-chloro-6-methyl-4-[3-(1,3-thiazol-2-yl)-1H-pyrazol-1-yl]-3-pyridinyl}ethanol

To a solution of intermediate 6 (903 mg, 2.84 mmol) in anh. MeOH (10 mL) were added CeCl₃ (700 mg, 1 eq) and NaBH₄ (107 mg, 1 eq) and the reaction mixture was stirred at room temperature for 5 min. The reaction was then quenched with water, extracted with ethyl acetate, washed with brine, dried over anh. Na₂SO₄, filtered and concentrated in vacuo to give 848 mg of the title compound as a white solid, which was used in the next step without further purification.

NMR (¹H, CDCl₃): δ 8.00 (m, 2H), 7.50 (d, 1H), 7.20 (m, 2H), 4.25 (t, 2H), 3.20 (t, 2H), 2.70 (s, 3H).

MS (m/z): 321 [MH]⁺.

Intermediate 8

### 2-chloro-3-(2-{[(1,1-dimethylethyl)(dimethyl)silyl]oxy}ethyl)-6-methyl-4-[3-(1,3-thiazol-2-yl)-1H-pyrazol-1-yl]pyridine

To a solution of intermediate 7 (840 mg, 2.6 mmol) in anh. CH₂Cl₂ (10 mL) were added 2,6-lutidine (0.67 ml, 2.2 eq) and tert-butyldimethylsilyl triflate (0.89 ml, 1.5 eq) and the reaction mixture was stirred at room temperature for 15 hr. The reaction was then quenched with an aqueous solution of saturated NH₄Cl, extracted with EtOAc, washed with brine, dried over anh. Na₂SO₄, filtered and concentrated in vacuo. The product was purified by flash chromatography (silica gel, cHex/EtOAc 3:2) to give 950 mg of the title compound as a colorless oil.

NMR (¹H, CDCl₃): δ 8.20 (d, 1H), 7.75 (d, 1H), 7.35 (d, 1H), 7.00 (m, 2H), 4.00 (t, 2H), 3.05 (t, 2H), 2.55 (s, 3H), 0.80 (s, 9H), −0.10 (s, 6H).

MS (m/z): 435 [MH]⁺.

Intermediate 9

### 3-(2-{[(1,1-dimethylethyl)(dimethyl)silyl]oxy}ethyl)-6-methyl-N-[6-(methyloxy)-2-(trifluoromethyl)-3-pyridinyl]-4-[3-(1,3-thiazol-2-yl)-1H-pyrazol-1-yl]-2-pyridinamine

To a solution of intermediate 8 (68 mg, 0.16 mmol) in anh. DME (2 mL) were added Pd₂(dba)₃ (15 mg, 0.1 eq), 2-(dicyclohexylphosphino)-2′-methylbiphenyl (17 mg, 0.3 eq), K₃PO₄ (90 mg, 3 eq) and intermediate 34 (37 mg, 1.2 eq) and the reaction mixture was submitted to microwave irradiation (150W, 100° C., 60 psi) for two 20 min cycles. The reaction was then quenched with an aqueous solution of saturated NH₄Cl, extracted with EtOAc, washed with brine, dried over anh. Na₂SO₄, filtered and concentrated in vacuo. The product was purified by flash chromatography (silica gel, CH₂Cl₂/MeOH 99:1) to give 22 mg of the title compound as a yellow foam.

NMR (¹H, CDCl₃): δ 8.40 (d, 1H), 7.85 (bs, 1H), 7.75 (bs, 1H), 7.31 (s, 1H), 7.03 (s, 1H), 6.90 (d, 1H), 6.63 (s, 1H), 4.16 (t, 2H), 3.94 (s, 3H), 2.76 (t, 2H), 2.36 (s, 3H), 0.77 (s, 9H), 0.04 (s, 6H).

Intermediate 10

### 3-(2-{[(1,1-Dimethylethyl)(dimethyl)silyl]oxy}ethyl)-6-methyl-N-[2-methyl-6-(methyoxy)-3-pyridinyl]-4-[3-(1,3-thiazol-2-yl)-1H-pyrazol-1-yl]-2-pyridinamine

As in intermediate 9, except that intermediate 75 (2-methyl-6-(methyloxy)-3-pyridinamine) was used instead of intermediate 34 (6-(Methyloxy)-2-(trifluoromethyl)-3-pyridinamine).

MS (m/z): 537 [MH]⁺.

Intermediate 11

### N-[2,6-Bis(methyloxy)-3-pyridinyl]-3-(2-{[(1,1-dimethylethyl)(dimethyl)silyl]oxy}ethyl)-6-methyl-4-[3-(1,3-thiazol-2-yl)-1H-pyrazol-1-yl]-2-pyridinamine

As in intermediate 9, except that 2,6-bis(methyloxy)-3-pyridinamine was used instead of intermediate 34 (6-(Methyloxy)-2-(trifluoromethyl)-3-pyridinamine).

NMR (¹H, CDCl₃): δ 8.70 (d, 1H); 7.86 (d, 1H); 7.75 (d, 1H); 7.32 (d, 1H); 7.03 (d, 1H); 6.60 (s, 1H); 6.32 (d, 1H); 4.15 (t, 2H); 3.99 (s, 3H); 3.88 (s, 3H); 2.47 (bs, 1H); 2.77 (t, 2H); 2.46 (s, 3H); 0.82 (s, 12H).

MS (m/z): 553 [MH]⁺.

Intermediate 12

### N⁵-{3-(2-{[1,1-Dimethylethyl)(dimethyl)silyl]oxy}ethyl)-6-methyl-4-[3-(1,3-thiazol-2-yl)-1H-pyrazol-1-yl]-2-pyridinyl}-N²,N²-trimethyl-2,5-pyridinediamine

As in intermediate 9, except that intermediate 36 (N²,N²,4-trimethyl-2,5-pyridinediamine) was used instead of intermediate 34 (6-(Methyloxy)-2-(trifluoromethyl)-3-pyridinamine).

NMR (¹H, DMSO-d₆): δ 8.35 (d, 1H), 8.00 (s, 1H), 7.99 (d, 1H), 7.87 (s, 1H), 7.83 (d, 1H), 7.07 (d, 1H), 6.69 (s, 1H), 6.63 (s, 1H), 3.95 (t, 2H), 3.10 (s, 6H), 2.98 (t, 2H), 2.28 (s, 3H), 2.18 (s, 3H), 0.85 (s, 9H), 0.00 (s, 6H).

MS (m/z): 550 [MH]⁺.

Intermediate 13

### N-[2-(Difluoromethyl)-4-(methyloxy)phenyl-3-(2-{[(1,1-dimethylethyl)(dimethyl)silyl]-oxy}ethyl)-6-methyl-4-[3-(1,3-thiazol-2-yl)-1H-pyrazol-1-yl]-2-pyridinamine

As in intermediate 9, except that intermediate 40 (2-(difluoromethyl)-4-(methyloxy)aniline) was used instead of intermediate 34 (6-(Methyloxy)-2-(trifluoromethyl)-3-pyridinamine).

NMR (¹H, CDCl₃): δ 7.9 (d, 1H), 7.8 (d, 1H), 7.68 (bs, 1H), 7.6 (d, 1H), 7.38 (d, 1H), 7.17 (d, 1H), 7.08 (d, 1H), 7.04 (dd, 1H), 6.77 (t, 1H; J_{(H-F)}=55.6 Hz), 6.64 (s, 1H), 4.24 (t, 2H), 3.89 (s, 3H), 2.81 (t, 2H), 2.41 (s, 3H), 0.88 (s, 9H), 0.00 (s, 6H).

MS (m/z): 572 [MH]⁺.

Intermediate 14

### N-[2-Chloro-4-(methyloxy)phenyl-3-(2-{[(1,1-dimethylethyl)(dimethyl)silyl]oxy}ethyl)-6-methyl-4-[3-(1,3-thiazol-2-yl)-1H-pyrazol-1-yl]-2-pyridinamine

As in intermediate 9, except that intermediate 41 (2-chloro-4-(methyloxy)aniline) was used instead of intermediate 34 (6-(Methyloxy)-2-(trifluoromethyl)-3-pyridinamine).

NMR (¹H, CDCl₃): δ 8.37 (d, 1H), 7.86 (d, 1H), 7.82 (bs, 1H), 7.77 (d, 1H), 7.32 (d, 1H), 7.03 (d, 1H), 6.95 (d, 1H), 6.84-6.82 (dd, 1H), 6.65 (s, 1H), 4.19 (t, 2H), 3.79 (s, 3H), 2.81 (t, 2H), 2.48 (s, 3H), 0.83 (s, 9H), 0.00 (s, 6H).

Intermediate 15

### N-[2,4-Bis(methyloxy)phenyl]-3-(2-{[(1,1-dimethylethyl)(dimethyl)silyl]oxy}ethyl)-6-methyl-4-[3-(1,3-thiazol-2-yl)-1H-pyrazol-1-yl]-2-pyridinamine

As in intermediate 9, except that 2,4-bis(methyloxy)aniline was used instead of intermediate 34 (6-(Methyloxy)-2-(trifluoromethyl)-3-pyridinamine).

NMR (¹H, CDCl₃): δ 8.21 (d, 1H), 7.84 (d, 1H), 7.75 (d, 1H), 7.34 (s, 1H), 7.31 (d, 1H), 7.02 (d, 2H), 6.50 (s, 1H), 3.96 (t, 2H), 3.78 (s, 3H), 3.72 (s, 3H), 3.05 (t, 2H), 2.55 (s, 3H), 1.81 (s, 9H), 0.00 (s, 6H).

MS (m/z): 553 [MH]⁺.

Intermediate 16

### 4-({[3-(2-{[1,1-dimethylethyl)(dimethyl)silyl]oxy}ethyl)-6-methyl-4-[3-(1,3-thiazol-2-yl)-1H-pyrazol-1-yl]-2-pyridinyl}amino)-3-[(trifluoromethyl)oxy]benzonitrile

As in intermediate 9, except that 4-amino-3-[(trifluoromethyl)oxy]benzonitrile was used instead of intermediate 34 (6-(Methyloxy)-2-(trifluoromethyl)-3-pyridinamine).

MS (m/z): 601 [MH]⁺.

Intermediate 17

### 4-({3-(2-{[(1,1-Dimethylethyl)(dimethyl)silyl]oxy}ethyl)-6-methyl-4-[3-(1,3-thiazol-2-yl)-1H-pyrazol-1-yl]-2-pyridinyl}amino)-3-ethylbenzonitrile

As in intermediate 9, except that 4-amino-3-ethylbenzonitrile was used instead of intermediate 34 (6-(Methyloxy)-2-(trifluoromethyl)-3-pyridinamine).

NMR (¹H, CDCl₃): δ 8.37 (bs, 1H), 8.07 (d, 1H), 7.87 (d, 1H), 7.77 (d, 1H), 7.43 (bs, 1H), 7.3 (d, 1H), 7.06 (d, 1H), 6.78 (s, 1H), 4.31 (m, 2H), 2.80 (m, 2H), 2.67 (q, 2H), 2.50 (s, 3H), 2.46 (m, 1H), 1.28 (t, 3H).

MS (m/z): 432 [MH]⁺.

Intermediate 18

### 3-(2-{[(1,1-Dimethylethyl)(dimethyl)silyl]oxy}ethyl)-N-(4-fluoro-2-methylphenyl)-6-methyl-4-[3-(1,3-thiazol-2-yl)-1H-pyrazol-1-yl-2-pyridinamine

As in intermediate 9, except that 4-fluoro-2-methylaniline was used instead of intermediate 34 (6-(Methyloxy)-2-(trifluoromethyl)-3-pyridinamine).

NMR (¹H, CDCl₃): δ 7.86 (s, 1H), 7.74 (d, 1H), 7.55 (d, 1H), 7.32 (s, 1H), 7.03 (s, 1H), 6.87 (m, 2H), 6.58 (s, 1H), 4.23 (t, 2H), 2.93 (bs, 1H), 2.76 (t, 2H), 2.40 (s, 3H), 2.24 (s, 3H), 0.83 (s, 9H), 0.02 (s, 6H).

MS (m/z): 524 [MH]⁺.

Intermediate 19

### 3-(2-{[(1,1-Dimethylethyl)(dimethyl)silyl]oxy}ethyl)-6-methyl-N-[2-methyl-4-(1H-pyrazol-1-yl)phenyl]-4-[3-(1,3-thiazol-2-yl)-1H-pyrazol-1-yl]-2-pyridinamine

As in intermediate 9, except that intermediate 43 (2-methyl-4-(1H-pyrazol-1-yl)aniline) was used instead of intermediate 34 (6-(Methyloxy)-2-(trifluoromethyl)-3-pyridinamine).

NMR (¹H, DMSO-d₆): δ 8.49 (dd, 1H), 8.36 (d, 1H), 8.03 (s, 1H), 7.99 (d, 1H), 7.84 (d, 1H), 7.77 (m, 2H), 7.70 (d, 1H), 7.65 (dd, 1H), 7.08 (d, 1H), 6.84 (s, 1H), 6.59 (m, 1H), 4.04 (t, 2H), 2.99 (t, 2H), 2.38 (s, 3H), 2.35 (s, 3H), 0.84 (s, 9H), 0.00 (s, 6H).

MS (m/z): 572 [MH]⁺.

Intermediate 20

### 3-(2-{[(1,1-Dimethylethyl)(dimethyl)silyl]oxy}ethyl)-N-[4-(nitro)-2-(trifluoromethyl)phenyl]-6-methyl-4-[3-(1,3-thiazol-2-yl)-1H-pyrazol-1-yl]-2-pyridinamine

As in intermediate 9, except that 4-nitro-2-(trifluoromethyl)aniline was used instead of intermediate 34 (6-(Methyloxy)-2-(trifluoromethyl)-3-pyridinamine).

NMR (¹H, CDCl₃): δ 8.37(s, 1H), 8.21(d, 1H), 8.18(d, 1H), 8.15(d, 1H), 7.87(d, 1H), 7.34(d, 1H), 7.03(d, 1H), 6.75(s, 1H), 6.72(s, 1H), 3.98(t, 2H), 3.08(t, 2H), 2.55(s, 3H), 1.55(s, 6H), 0.75(s, 9H).

MS (m/z): 605 [MH]⁺.

Intermediate 21

### 2-{6-Methyl-2-{[6-(methyloxy)-2-(trifluoromethyl)-3-pyridinyl]amino}-4-[3-(1,3-thiazol-2-yl)-1H-pyrazol-1-yl]-3-pyridinyl}ethanol

To a solution of intermediate 9 (240 mg, 0.38 mmol) in anh. THF (5 mL) was added Et₃N.3HF (0.187 ml, 3 eq) and the reaction mixture was stirred for 15 hr at r.t. The reaction was then quenched with sta.aq. NH₄Cl, extracted with EtOAc, washed with sat.aq.NaCl, dried over anh. Na₂SO₄, filtered and concentrated in vacuo. The product was purified by flash chromatography (silica gel, cHex/EtOAc 1:1) to give 180 mg of the title compound as a colorless oil.

NMR (¹H, CDCl₃): δ 8.45 (bs, 1H), 8.20 (d, 1H), 7.85 (d, 1H), 7.85 (2d, 2H), 7.65 (dd, 1H), 7.30 (d, 1H), 7.05 (d, 1H), 6.85 (s, 1H), 4.20 (t, 2H), 2.85 (t, 2H), 2.50 (s, 3H).

MS (m/z): 514 [MH]⁺.

Intermediate 22

### 2-{6-Methyl-2-{[6-methyl-2-(methyloxy)-3-pyridinyl]amino}-4-[3-(1,3-thiazol-2-yl)-1H-pyrazol-1-yl]-3-pyridinyl}ethanol

As in intermediate 21, except that intermediate 10 was used instead of intermediate 9.

MS (m/z): 423 [MH]⁺.

Intermediate 23

### 2-{2-{[2,6-Bis(methyloxy)-3-pyridinyl]amino}-6-methyl-4-[3-(1,3-thiazol-2-yl)-1H-pyrazol-1-yl]-3-pyridinyl}ethanol

As in intermediate 21, except that intermediate 11 was used instead of intermediate 9.

NMR (¹H, CDCl₃): δ 8.60 (d, 1H); 7.88 (d, 1H); 7.74 (d, 1H); 7.31 (d, 1H); 7.02 (d, 1H); 6.56 (s, 1H); 6.32 (d, 1H); 4.13 (t, 2H); 3.97 (s, 3H); 3.86 (s, 3H); 3.78 (t, 1H); 2.77 (t, 2H); 2.46 (s, 3H).

MS (m/z): 439 [MH]⁺.

Intermediate 24

### 2-{2-{[6-(Dimethylamino)-4-methyl-3-pyridinyl]amino}-6-methyl-4-[3-(1,3-thiazol-2-yl)-1H-pyrazol-1-yl]-3-pyridinyl}ethanol

As in intermediate 21, except that intermediate 12 was used instead of intermediate 9.

NMR (¹H, DMSO-d₆): δ 8.26 (d, 1H), 8.02 (s, 1H), 7.97 (s, 1H), 7.90 (d, 1H), 7.73 (d, 1H), 6.98 (d, 1H), 6.62 (s, 1H), 6.53 (s, 1H), 5.16 (t, 1H), 3.81 (q, 2H), 3.00 (s, 6H), 2.21 (s, 3H), 2.10 (s, 3H).

MS (m/z): 436 [MH]⁺.

Intermediate 25

### 2-{2-{[2-(Difluoromethyl)-4-(methyloxy)phenyl]amino}-6-methyl-4-[3-(1,3-thiazol-2-yl)-1H-pyrazol-1-yl]-3-pyridinyl}ethanol

As in intermediate 21, except that intermediate 13 was used instead of intermediate 9.

NMR (¹H, CDCl₃): δ 7.88 (d, 1H), 7.76 (d, 1H), 7.67 (d, 1H), 7.48 (bs, 1H), 7.34 (d, 1H), 7.09 (d, 1H), 7.06 (d, 1H), 7.01 (dd, 1H), 6.74 (t, 1H; J_{(H-F)}=55.3 Hz), 6.62 (s, 1H), 4.21 (m, 2H), 3.85 (s, 3H), 3.18 (t, 1H), 2.83 (t, 2H), 2.4 (s, 3H).

MS (m/z): 458 [MH]⁺.

Intermediate 26

### 2-{2-{[2-Chloro-4-(methyloxy)phenyl]amino}-6-methyl-4-[3-(1,3-thiazol-2-yl)-1H-pyrazol-1-yl]-3-pyridinyl}ethanol

As in intermediate 21, except that intermediate 14 was used instead of intermediate 9.

NMR (¹H, CDCl₃): δ 8.27 (d, 1H), 7.86 (d, 1H), 7.58 (bs, 1H), 7.33 (d, 1H), 7.57 (d, 1H), 7.05 (d, 1H), 6.95 (d, 1H), 6.84-6.82 (dd, 1H), 6.64 (s, 1H), 4.20 (t, 2H), 3.78 (s, 3H), 3.49 (t, 1H), 2.8 (t, 2H), 2.48 (s, 3H).

Intermediate 27

### 2-{2-{[2,4-Bis(methyloxy)phenyl]amino}-6-methyl-4-[3-(1,3-thiazol-2-yl)-1H-pyrazol-1-yl]-3-pyridinyl}ethanol

As in intermediate 21, except that intermediate 15 was used instead of intermediate 9.

NMR (¹H, CDCl₃): δ 8.35 (d, 1H), 7.85 (d, 1H), 7.73 (d, 1H), 7.40 (s, 1H), 7.31 (d, 1H), 7.05 (d, 1H), 6.52 (s, 1H), 6.50 (s, 1H), 4.18 (m, 2H), 3.87 (s, 3H), 3.80 (s, 3H), 2.85 (t, 2H), 2.48 (s, 3H).

MS (m/z): 440 [MH]⁺.

Intermediate 28

### 4-({3-(2-hydroxyethyl)-6-methyl-4-[3-(1,3-thiazol-2-yl)-1H-pyrazol-1-yl]-2-pyridinyl}amino)-3-[(trifluoromethyl)oxy]benzonitrile

As in intermediate 21, except that intermediate 16 was used instead of intermediate 9.

NMR (¹H, CDCl₃): δ 9.27 (s, 1H); 8.45 (d, 1H); 7.90 (d, 1H); 7.80 (d, 1H); 7.50 (dd, 2H); 7.30 (d, 1H); 7.10 (d, 1H); 6.95 (d, 1H); 4.45 (t, 2H); 2.80 (t, 2H); 2.50 (s, 3H); 2.45 (s, 1H).

MS (m/z): 487 [MH]⁺.

Intermediate 29

### 3-ethyl-4-({3-(2-hydroxyethyl)-6-methyl-4-[3-(1,3-thiazol-2-yl)-1H-pyrazol-1-yl]-2-pyridinyl}amino)benzonitrile

As in intermediate 21, except that intermediate 17 was used instead of intermediate 9.

NMR (¹H, CDCl₃): δ 8.37 (bs, 1H), 8.07 (d, 1H), 7.87 (d, 1H), 7.77 (d, 1H), 7.43 (bs, 1H), 7.3 (d, 1H), 7.06 (d, 1H), 6.78 (s, 1H), 4.31 (m, 2H), 2.80 (m, 2H), 2.67 (q, 2H), 2.50 (s, 3H), 2.46 (m, 1H), 1.28 (t, 3H).

MS (m/z): 414 [MH]⁺.

Intermediate 30

### 2-{2-[(4-fluoro-2-methylphenyl)amino]-6-methyl-4-[3-(1,3-thiazol-2-yl)-1H-pyrazol-1-yl]-3-pyridinyl}ethanol

As in intermediate 21, except that intermediate 18 was used instead of intermediate 9.

NMR (¹H, CDCl₃): δ 7.92 (d, 1H), 7.70 (s, 1H), 7.65 (d, 1H), 7.50 (s, 1H), 7.32 (s, 1H), 7.00 (s, 1H), 6.80-6.95 (m, 2H), 6.58 (s, 1H), 4.25 (bs, 2H), 2.93 (bs, 1H), 2.83 (t, 2H), 2.40 (s, 3H), 2.27 (s, 3H).

MS (m/z): 410 [MH]⁺.

Intermediate 31

### 2-{6-Methyl-2-{[2-methyl-4-(1H-pyrazol-1-yl)phenyl]amino}-4-[3-(1,3-thiazol-2-yl)-1H-pyrazol-1-yl]-3-pyridinyl}ethanol

As in intermediate 21, except that intermediate 19 was used instead of intermediate 9.

NMR (¹H, DMSO-d₆): δ 8.58 (s, 1H), 8.38 (dd, 1H), 8.28 (d, 1H), 7.90 (d, 1H), 7.74 (m, 1H), 7.68 (dd, 1H), 7.66 (d, 1H), 7.57 (dd, 1H), 7.00 (d, 1H), 6.81 (s, 1H), 6.49 (m, 1H), 5.61 (bs, 1H), 3.94 (m, 2H), 2.70 (m, 2H), 2.33 (s, 3H), 2.28 (s, 3H).

MS (m/z): 458 [MH]⁺.

Intermediate 32

### 2-{2-{[4-nitro-2-(trifluoromethyl)phenyl]amino}-6-methyl-4-[3-(1,3-thiazol-2-yl)-1H-pyrazol-1-yl]-3-pyridinyl}ethanol

As in intermediate 21, except that intermediate 20 was used instead of intermediate 9.

NMR (¹H, CDCl₃): δ 9.05(s, 1H), 8.50(d, 1H), 8.25(d, 1H), 8.15(dd, 1H), 7.85(d, 1H), 7.75(d, 1H), 7.32(d, 1H), 7.05(d, 1H), 6.90(s, 1H), 4.20(m, 2H), 2.80(t, 2H), 2.60(bs, 1H), 2.55(s, 3H).

MS (m/z): 491 [MH]⁺.

Intermediate 33

### 6-(methyloxy)-3-nitro-2-(trifluoromethyl)pyridine

To a solution of 2-chloro-3-nitro-5-methoxypyridine (500 mg, 2.6 mmol) in DMA (5 mL), at r.t., under N₂, were added Cu (1 g, 6 eq) and CF₂Br₂ (500 μL, large excess). The reaction mixture was stirred at 100° C. for 8 hr. The brown slurry obtained was extracted with Et₂O (3×50 mL). The combined organic extracts were dried under vacuum and the crude product was purified by flash chromatography (silica gel, CH₂Cl₂ 100%) to give the title compound (316 mg, 53%) as a brown oil.

NMR (¹H, CDCl₃): δ 8.35 (d, 1H), 7.2 (d,1H), 4.3 (s,3H).

Intermediate 34

### 6-(Methyloxy)-2-(trifluoromethyl)-3-pyridinamine

To a solution of intermediate 33 (98 mg, 0.44 mmol) in MeOH (10 mL), at r.t., was added Pd/C 10% (30 mg 30% w/w) and the resulting suspension subjected to hydrogenation (1 atm) for 2 hr. The palladium was filtered off and the solvent was evaporated under reduced pressure. The crude product was purified by flash chromatography (silica gel, CH₂Cl₂/MeOH 95:5) to give the title compound (37 mg, 44%) as a red oil.

NMR (¹H, CDCl₃): δ 7.10 (d, 1H); 6.56 (d,1H); 3.85 (s,3H); 3.82-3.55 (bs, 2H).

Intermediate 35

### N,N,4-Trimethyl-5-nitro-2-pyridinamine

To 2-chloro-4-methyl-5-nitropyridine (2.78 g, 16.1 mmol), at r.t., under N₂, was added Me₂NH 2N/THF (55.4 mL). The reaction mixture was heated at 80° C. for 2 hr. It was cooled down to r.t. and partitioned between CH₂Cl₂ and water. The phases were separated and the aqueous layer was further extracted with CH₂Cl₂ (3×30 mL). The combined organic extracts were dried over anh. Na₂SO₄, the solids were filtered and the solvent evaporated. The crude title compound was used in the next step without further purification (2.97 g, quantitative yield).

NMR (¹H, CDCl₃): δ 8.99 (s, 1H), 6.24 (s, 1H), 3.2 (s, 6H), 2.62 (s, 3H).

MS (m/z): 182 [MH]⁺.

Intermediate 36

### N²,N²,4-Trimethyl-2,5-pyridinediamine

To a suspension of intermediate 35 (2.97 g, 16.31 mmol) in a 1:1 mixture of MeOH/H₂O (100 mL), at r.t., under N₂, were added Fe (3.19 g, 3.5 eq) and NH₄Cl (3.04 g, 3.5 eq). The reaction mixture was heated at 80° C. for 1.5 hr. The mixture was filtered and the solid was washed with MeOH. The crude was evaporated to dryness and partitioned between CH₂Cl₂ and water. The phases were separated and the aqueous layer was further extracted with CH₂Cl₂ (3×30 mL). The combined organic extracts were dried over anh. Na₂SO₄, the solids were filtered and the solvent evaporated. The crude product was purified by flash-chromatography (silica gel, EtOAc/MeOH 9:1-7:3) to give the title compound as a yellow solid (340 mg, 14%)

NMR (¹H, DMSO-d₆): δ 7.52 (s, 1H), 6.36 (s, 1H), 4.17 (bs, 2H), 2.83 (s, 6H), 2.03 (s, 3H). MS (m/z): 152 [MH]⁺.

Intermediate 37

### (5-Methoxy-2-nitro-phenyl)-methanol

To a suspension of cyanuric chloride (1.84 g, 1 eq) in anh. DME (60 mL) at r.t., under N₂, was added NMM (1.1 mL, 1 eq). The reaction mixture was stirred for 2 min and a precipitate was formed. A solution of 5-(methyloxy)-2-nitrobenzoic acid (2.0 g, 10 mmol) in anh. DME (20 mL) was added and the reaction mixture was stirred for 4 hr. The suspension was filtered and a solution of NaBH₄ (0.57 g, 1.5 eq) in water (30 mL) was added at 0° C. The reaction mixture was stirred for 20 min at 0° C. It was then diluted with Et₂O (10 mL) and acidified to pH=5 by addition of sat. aq. NH₄Cl. The phases were separated and the aqueous layer was extracted with Et₂O (2×100 mL). The combined organic extracts were washed with sat. aq. Na₂CO₃ and dried over anh. Na₂SO₄. The solids were filtered and the solvent evaporated to dryness. The crude product was purified by flash chromatography (silica gel, cHex/EtOAc 8:2) to give the title compound (958 mg, 53%).

NMR (¹H, CDCl₃): δ 8.15 (d, 1H), 7.19 (m, 1H), 6.85 (dd, 1H), 4.95 (d, 2H), 3.89 (s, 3H), 2.5 (t, 1H).

Intermediate 38

### 5-(Methyloxy)-2-nitrobenzaldehyde

To a solution of intermediate 37 (1.44 g, 7.9 mmol) in anh. CH₂Cl₂ (40 mL) at r.t., under N₂, was added Dess-Martin periodinane (3.68 g, 1.1 eq). The reaction mixture was stirred for 3 hr at r.t., then sat.aq. Na₂S₂O₃ (5 mL) and sat.aq. NaHCO₃ (20 mL) were added. The phases were separated and the aqueous layer was extracted with EtOAc (2×100 mL). The combined organic extracts were dried over anh. Na₂SO₄, the solids were filtered and the solvent evaporated to dryness to give 1.45 g (100%) of the title compound.

NMR (¹H, CDCl₃): δ 10.47 (s, 1H), 8.14 (d, 1H), 7.31 (d, 1H), 7.13 (dd, 1H), 3.94 (s, 3H).

Intermediate 39

### 2-(Difluoromethyl)-4-(methyloxy)-1-nitrobenzene

To a solution of intermediate 38 (250 mg, 1.38 mmol) in anh. CH₂Cl₂ (10 mL), at −78° C., under N₂, was added slowly DAST (2×0.4 mL, 2.2 eq). The reaction mixture was stirred at r.t. for 1.5 hr, after which was added sat.aq. NaCl. The phases were separated and the aqueous layer was extracted with EtOAc (3×20 mL). The combined organic extracts were dried over anh. Na₂SO₄, the solids were filtered and the solvent was evaporated to dryness. The crude product was purified by flash chromatography (silica gel, cHex/EtOAc 8:2) to give the title compound (176 mg, 63%) as a yellow oil.

NMR (¹H, CDCl₃): δ 8.21 (d, 1H), 7.43 (t, 1H; J_{(H-F)}=54.9 Hz), 7.33 (d, 1H), 7.06 (dd, 1H), 3.95 (s, 3H).

Intermediate 40

### 2-(Difluoromethyl)-4-(methyloxy)aniline

To a solution of intermediate 39 (176 mg, 0.87 mmol) in anh. MeOH (8.7 mL), at r.t., under N₂, was added Pd/C 10% (88 mg, 5% wt). The reaction mixture was placed under an atmosphere of H₂ for 5 hr. The catalyst was filtered off and the solution obtained was evaporated to dryness. The crude product was purified by flash chromatography (silica gel, cHex/EtOAc 9:1) to give the title compound (27 mg, 20%) as a yellow oil.

NMR (¹H, CDCl₃): δ 6.88-6.80 (m, 2H); 6.7-6.67 (m, 1H); 6.62 (t, 1H; J_{(H-F)}=55.6 Hz); 3.8-3.5 (bs, 2H); 3.76 (s, 3H)

MS (m/z): 174 [MH]⁺.

Intermediate 41

### 2-Chloro-4-(methyloxy)aniline

To a suspension of 4-amino-3-chlorophenol hydrochloride (500 mg, 2.7 mmol) in acetone (5 mL), at r.t., under N₂, were added K₂CO₃ (138 mg, 1 eq) and t-BuOK (311 mg, 1 eq). MeI was then added (173 μL, 1 eq.). The reaction mixture was stirred at r.t. for 2 hr. CH₂Cl₂ (10 mL) was added and the reaction quenched with H₂O (5 mL). The phases were separated and the aqueous layer was extracted with CH₂Cl₂ (5 mL). The combined organic extracts were dried over anh. Na₂SO₄, the solids were filtered and the solvent evaporated under reduced pressure. The crude product was purified by flash chromatography (silica gel, CH₂Cl₂ 100%) to give the title compound (180 mg, 41%).

NMR (¹H, DMSO): δ 6.81 (d, 1H), 6.72 (s,1H), 6.68 (d,1H), 4.81 (s,2H), 3.63 (s, 3H).

Intermediate 42

### 1,1-Dimethylethyl (4-bromo-2-methylphenyl)carbamate

To a solution of 4-bromo-2-methylaniline (1 g, 5.37 mmol) in 1,4-dioxane (11 mL) and H₂O (4 mL), at r.t., were added Et₃N (2.7 mL, 1.2 eq) and BOC₂O (4.2 g, 1.2 eq). The reaction mixture was stirred at r.t. for 96 hr. Sat.aq. NH₄Cl and EtOAc (20 mL) were added and the phases were separated. The aqueous layer was further extracted with EtOAc (2×20 mL). The combined organic extracts were dried over anh. Na₂SO₄, the solids were filtered and the solvent evaporated. The residue was purified by SCX Column (Eluents: CH₂Cl₂, MeOH and NH₃(0.5 M in MeOH)) to give the title compound as a white solid (1.22 g, 79%).

NMR (¹H, DMSO-d₆): δ 8.55 (s, 1H), 7.35 (m, 1H), 7.28 (m, 2H), 2.17 (s, 3H), 1.44 (s, 9H).

MS (m/z): 230 [MH-tBu]⁺; 186 [MH-BOC]⁺.

Intermediate 43

### 2-Methyl-4-(1H-pyrazol-1-yl)aniline

A solution of intermediate 42 (200 mg, 0.7 mmol), 1H-pyrazole (95 mg, 2 eq), CuI (133 mg, 1 eq), K₂CO₃ (290 mg, 2.1 eq) and (1R,2R)-diaminomethylcyclohexane (100 mg, 1 eq) in anh. NMP (1 mL), under N₂, was heated at 150° C. for 6 h. It was cooled down to r.t. and poured into water. EtOAc was added and the phases were separated. The aqueous layer was further extracted with EtOAc (2×10 mL). The combined organic extracts were dried over anh. Na₂SO₄, the solids were filtered and the solvent evaporated. The residue was purified by flash-chromatography (silica gel, cHex/EtOAc 8:2) to give the title compound as a white solid (85.6 mg, 70%).

NMR (¹H, CDCl₃): δ 7.77 (dd, 1H), 7.66 (d, 1H), 7.39 (d, 1H), 7.29 (dd, 1H), 6.72 (d, 1H), 6.40 (t, 1H), 2.85 (bs, 1H).

MS (m/z): 174 [MH]⁺.

Intermediate 44

### Ethyl 4-(1′H-1,3′-bipyrazol-1′-yl)-2-chloro-6-methyl-3-pyridinecarboxylate

As in intermediate 2, except that intermediate 58 was used instead of 2-(1H-pyrazol-3-yl)-1,3-thiazole.

NMR (¹H, CDCl₃): δ 8.10 (s, 1H), 7.90 (d, 1H), 7.68 (s, 1H), 7.29 (s, 1H), 6.85 (d, 1H), 6.43 (s,1H), 4.40 (t, 2H), 2.57 (s, 3H), 1.24 (q, 3H).

MS (m/z): 332 [MH]⁺.

Intermediate 45

### [4-(1′H-1,3′-Bipyrazol-1′-yl)-2-chloro-6-methyl-3-pyridinyl]methanol

As in intermediate 3, except that intermediate 44 was used instead of intermediate 2.

NMR (¹H, CDCl₃): δ 7.12 (s, 1H), 7.99 (d, 1H), 7.74 (s, 1H), 7.23 (s, 1H), 6.84 (d, 1H), 6.44 (s, 1H), 4.76 (bs, 2H), 3.74 (bs, 1H), 2.60 (s, 3H).

MS (m/z): 290 [MH]⁺.

Intermediate 46

### 4-(1′H-1,3′-Bipyrazol-1′-yl)-2-chloro-6-methyl-3-pyridinecarbaldehyde

As in intermediate 4, except that intermediate 45 was used instead of intermediate 3.

NMR (¹H, CDCl₃): δ 10.45 (s, 1H), 8.13 (s, 1H), 7.94 (s, 1H), 7.71 (s, 1H), 7.33 (s, 1H), 6.82 (s, 1H), 6.41 (s, 1H), 2.67 (s, 3H).

MS (m/z): 288 [MH]⁺.

Intermediate 47

### 1′-{2-Chloro-6-methyl-3-[(E)-2-(methyloxy)ethenyl]-4-pyridinyl}-1′H-1,3′-bipyrazole

As in intermediate 5, except that intermediate 46 was used instead of intermediate 4.

NMR (¹H, CDCl₃): δ 8.1-7.8 (m, 2H), 7.7-7.6 (m, 2H), 7.5-7.2 (m, 1H), 7.0-6.8 (s, 1H), 6.1 (d, 1H), 5.4 (s, 1H), 3.40 (s, 3H), 2.60 (s, 3H).

MS (m/z): 316 [MH]⁺.

Intermediate 48

### [4-(1′H-1,3′-Bipyrazol-1′-yl)-2-chloro-6-methyl-3-pyridinyl]acetaldehyde

As in intermediate 6, except that intermediate 47 was used instead of intermediate 5

MS (m/z): 302 [MH]⁺.

Intermediate 49

### 2-[4-(1′H-1,3′-Bipyrazol-1′-yl)-2-chloro-6-methyl-3-pyridinyl]ethanol

As in intermediate 7, except that intermediate 48 was used instead of intermediate 6

NMR (¹H, CDCl₃): δ 7.9 (d, 1H), 8.0 (d, 1H), 7.4 (t, 1H), 7.1 (s, 1H), 6.8 (d, 2H), 4.2 (t, 2H), 3.90 (tb, 1H), 3.2 (t, 2H), 2.40 (s, 3H).

MS (m/z): 304 [MH]⁺.

Intermediate 50

### 1′-[2-Chloro-3-(2-{[(1,1-dimethylethyl)(dimethyl)sily]oxy}ethyl)-6-methyl-4-pyridinyl]-1′H-1,3′-bipyrazole

As in intermediate 8, except that intermediate 49 was used instead of intermediate 7

NMR (¹H, CDCl₃): δ 8.4 (d, 1H), 8.2 (d, 1H), 8.15 (d, 1H), 7.8 (d, 1H), 6.8 (d, 1H), 6.4 (s, 1H), 4,10 (t, 2H), 3.15 (t, 2H), 2.50 (s, 3H), 0.95 (s, 9H), 0.1 (s, 6H).

MS (m/z): 418 [MH]⁺.

Intermediate 51

### 4-{[4-(1′H-1,3′-Bipyrazol-1′-yl)-3-(2-{[(1,1-dimethylethyl)(dimethyl)silyl]oxy}ethyl)-6-methyl-2-pyridinyl]amino}-3-methylbenzonitrile

As in intermediate 9, except that intermediate 50 was used instead of intermediate 8, and 4-amino-3-methylbenzonitrile was used instead of intermediate 34.

NMR (¹H, CDCl₃): δ 8.60 (d, 1H), 8.15 (m, 2H), 7.75 (m, 2H), 7.50 (d, 1H), 7.20 (d, 2H), 6.80 (d, 2H), 6.4 (d, 1H), 4.0 (t, 2H), 3.05 (t, 2H), 2,60 (s, 3H), 2.20 (s, 3H), 0.90 (s, 9H), 0.10 (s, 6H).

MS (m/z): 514 [MH]⁺.

Intermediate 52

### 4-{[4-(1′H-1,3′-Bipyrazol-1′-yl)-3-(2-{[(1,1-dimethylethyl)(dimethyl)silyl]oxy}ethyl)-6-methyl-2-pyridinyl]amino}-3-(trifluoromethyl)benzonitrile

As in intermediate 9, except that intermediate 50 was used instead of intermediate 8, and 4-amino-3-(trifluoromethyl)benzonitrile was used instead of intermediate 34.

R_{f}=0.25 (7:3 cHx/EtOAc)

Intermediate 53

### 4-{[4-(1′H-1,3′-Bipyrazol-1′-yl)-3-(2-{[(1,1-dimethylethyl)(dimethyl)silyl]oxy}ethyl)-6-methyl-2-pyridinyl]amino}-3-chlorobenzonitrile

As in intermediate 9, except that intermediate 50 was used instead of intermediate 8, and 4-amino-3-chlorobenzonitrile was used instead of intermediate 34.

NMR (¹H, CDCl₃): δ 8.60 (d, 1H), 8.15 (m, 2H), 7.75-7.65 (m, 2H), 7.50 (d, 1H), 7.20 (d, 2H), 6.80 (d, 2H), 6.4 (d, 1H), 3.90 (t, 2H), 3.15 (t, 2H), 2.20 (s, 3H), 0.90 (s, 9H), 0.10 (s, 6H).

MS (m/z): 535 [MH]⁺.

Intermediate 54

### 4-{[4-(1′H-1,3′-Bipyrazol-1′-yl)-3-(2-hydroxyethyl)-6-methyl-2-pyridinyl]amino}-3-methylbenzonitrile

As in intermediate 21, except that intermediate 51 was used instead of intermediate 9

NMR (¹H, CDCl₃): δ 8.40 (s, 1H), 8.15 (m, 2H), 7.65 (m, 2H), 7.40 (m, 2H), 6.8 (m, 2H), 6.4 (d,1H), 4.2 (t, 2H), 2.85 (t, 2H), 2,60 (t, 1H), 2.50 (s, 3H), 2.20 (s, 3H).

MS (m/z): 400 [MH]⁺.

Intermediate 55

### 4-{[4-(1′H-1,3′-Bipyrazol-1′-yl)-3-(2-hydroxyethyl)-6-methyl-2-pyridinyl]amino}-3-(trifluoromethyl)benzonitrile

As in intermediate 21, except that intermediate 52 was used instead of intermediate 9

NMR (¹H, CDCl₃): δ 8.25 (s, 1H), 8.10 (m, 2H), 7.55 (m, 2H), 7.40 (m, 2H), 7.0 (m, 2H), 6.5 (d,1H), 4.2 (t, 2H), 2.85 (t, 2H), 2,60 (t, 1H), 2.50 (s, 3H).

MS (m/z): 454 [MH]⁺.

Intermediate 56

### 4-{[4-(1′H-1,3′-Bipyrazol-1′-yl)-3-(2-hydroxyethyl)-6-methyl-2-pyridinyl]amino}-3-chlorobenzonitrile

As in intermediate 21, except that intermediate 53 was used instead of intermediate 9

NMR (¹H, CDCl₃): δ 8.35 (s, 1H), 8.15 (m, 2H), 7.65-7.55 (m, 2H), 7.40 (m, 2H), 6.8 (m, 2H), 6.4 (d,1H), 4.2 (t, 2H), 2.85 (t, 2H), 2,60 (t, 1H), 2.50 (s, 3H).

MS (m/z): 420 [MH]⁺.

Intermediate 57

### 1,2-Dihydro-3H-pyrazol-3-one hydrazone hydrochloride

To a solution of 1H-pyrazol-3-amine (3 g, 1 eq) in 6N HCl (22 mL), at −5° C., was added a 1M aqueous solution of NaNO₂ (36 mL, 1 eq). A solution of SnCl₂ (13.7 g, 2 eq) in conc. HCl (62 mL) was then added dropwise and the resulting reaction mixture was stirred at r.t. for 2 hr. The solvents were evaporated to give 13 g of a brown solid which was used in the next step without further purification.

Intermediate 58

### 1′H-1,3′-Bipyrazole

Intermediate 57 was dissolved in a water/ethanol solution (40 mL/28 mL). 1,1,3,3-tetramethoxypropane (8.7 mL, 1.1 eq) was then added and the resulting reaction mixture was stirred for 18 hr at r.t. The solution was neutralized with NaHCO₃ and K₂CO₃, extracted with EtOAc (200 mL) and CH₂Cl₂ (200 mL). The combined organic extracts were dried over anh. Na₂SO₄, the solids were filtered and the solvent evaporated. The crude product was purified by flash chromatography (silica gel, cHex/EtOAc 3:7) to give the title compound as a yellow solid (3.6 g, 74%).

NMR (¹H, DMSO-d₆): δ 12.45 (s, 1H), 8.25 (bs, 1H), 7.83 (bs, 1H), 7.67 (bs, 1H), 6.45 (bs, 2H).

MS (m/z): 134 [MH]⁺.

Intermediate 59

### Ethyl 2-chloro-6-methyl-4-[3-(2-pyridinyl)-1H-pyrazol-1-yl]-3-pyridinecarboxylate

As in intermediate 2, except that intermediate 72 (2-(1H-pyrazol-3-yl)pyridine) was used instead of 2-(1H-pyrazol-3-yl)-1,3-thiazole.

NMR (¹H, CDCl₃): δ 8.56 (d, 1H), 7.93 (d, 1H), 7.88 (d, 1H), 7.68 (dt, 1H), 7.17 (m, 2H), 7.11 (d, 1H), 4.30 (q, 2H), 2.54 (s, 3H), 1.18 (t, 3H).

MS (m/z): 343 [MH]⁺.

Intermediate 60

### {2-Chloro-6-methyl-4-[3-(2-pyridinyl)-1H-pyrazol-1-yl]-3-pyridinyl}methanol

As in intermediate 3, except that intermediate 59 was used instead of intermediate 2.

NMR (¹H, CDCl₃): δ 8.67 (d, 1H), 8.00 (d, 1H), 7.93 (d, 1H), 7.77 (dt, 1H), 7.25-7.19 (m, 3H), 4.77 (d, 2H), 4.44 (t, 1H), 2.59 (s, 3H).

MS (m/z): 301 [MH]⁺.

Intermediate 61

### 2-Chloro-6-methyl-4-[3-(2-pyridinyl)-1H-pyrazol-1-yl]-3-pyridinecarbaldehyde

As in intermediate 4, except that intermediate 60 was used instead of intermediate 3.

NMR (¹H, CDCl₃): δ 10.33 (s, 1H), 8.64 (d, 1H), 8.01 (d, 1H), 7.99 (d, 1H), 7.77 (dt, 1H), 7.34-7.22 (m, 3H), 2.66 (s, 3H).

MS (m/z): 299 [MH]⁺.

Intermediate 62

### 2-Chloro-6-methyl-3-[(E)-2-(methyloxy)ethenyl]-4-[3-(2-pyridinyl)-1H-pyrazol-1-yl]pyridine

As in intermediate 5, except that intermediate 61 was used instead of intermediate 4.

NMR (¹H, CDCl₃): δ 8.65 (dd, 1H), 8.02 (dd, 1H), 7.82 (d, 1H), 7.72 (dt, 1H), 7.36 (s, 1H), 7.26 (m, 1H), 7.09 (d, 1H), 6.48 (d, 1H), 5.61 (d, 1H), 3.60 (s, 3H), 2.54 (s, 3H).

MS (m/z): 327 [MH]⁺.

Intermediate 63

### {2-Chloro-6-methyl-4-[3-(2-pyridinyl)-1H-pyrazol-1-yl]-3-pyridinyl}acetaldehyde

As in intermediate 6, except that intermediate 62 was used instead of intermediate 5.

NMR (¹H, CDCl₃): δ 9.82 (s, 1H), 8.62 (d, 1H), 7.92 (d, 1H), 7.81 (d, 1H), 7.74 (dt, 1H), 7.27-7.16 (m, 3H), 4.08 (s, 2H). 2.60 (s, 3H).

MS (m/z): 313 [MH]⁺.

Intermediate 64

### 2-{2-Chloro-6-methyl-4-[3-(2-pyridinyl)-1H-pyrazol-1-yl]-3-pyridinyl}ethanol

As in intermediate 7, except that intermediate 63 was used instead of intermediate 6.

NMR (¹H, CDCl₃): δ 8.65 (d, 1H), 7.95 (dd, 1H), 7.82 (d, 1H), 7.76 (dt, 1H), 7.27 (m, 1H), 7.19 (d,1H), 7.09 (s, 1H), 5.07 (bs, 1H), 4.11 (t, 2H), 3.09 (t, 2H), 2.57 (s, 3H).

MS (m/z): 315 [MH]⁺.

Intermediate 65

### 2-chloro-3-(2-{[(1,1-dimethylethyl)(dimethyl)silyl]oxy}ethyl)-6-methyl-4-[3-(2-pyridinyl)-1H-pyrazol-1-yl]pyridine

As in intermediate 8, except that intermediate 64 was used instead of intermediate 7.

NMR (¹H, CDCl₃): δ 8.64 (dd, 1H), 8.17 (dd, 1H), 8.04 (d, 1H), 7.72 (dt, 1H), 7.24 (m, 2H), 7.12 (d, 1H), 3.99 (t, 2H), 3.10 (t, 2H), 2.55 (s, 3H), 0.79 (s, 9H), 0.05 (s, 6H).

MS (m/z): 429 [MH]⁺.

Intermediate 66

### 4-({3-(2-{[(1,1-Dimethylethyl)(dimethyl)silyl]oxy}ethyl)-6-methyl-4-[3-(2-pyridinyl)-1H-pyrazol-1-yl]-2-pyridinyl}amino)-3-methylbenzonitrile

As in intermediate 9, except that intermediate 65 was used instead of intermediate 8, and 4-amino-3-methylbenzonitrile was used instead of intermediate 34.

NMR (¹H, CDCl₃): δ 8.64-6.59 (m, 7H), 4.23 (t, 2H), 4.07 (bs, 1H), 2.83 (t, 2H), 2.53 (s, 3H), 2.32 (s, 3H), 0.81 (s, 9H), 0.03 (s, 6H).

MS (m/z): 525 [MH]⁺.

Intermediate 67

### 3-Chloro-4-({3-(2-{[(1,1-dimethylethyl)(dimethyl)silyl]oxy}ethyl)-6-methyl-4-[3-(2-pyridinyl)-1H-pyrazol-1-yl]-2-pyridinyl}amino)benzonitrile

As in intermediate 9, except that intermediate 65 was used instead of intermediate 8, and 4-amino-3-chlorobenzonitrile was used instead of intermediate 34.

NMR (¹H, CDCl₃): δ 8.64 (d, 1H), 8.59 (d, 1H), 8.17 (s, 1H), 8.05 (d, 1H), 7.75 (d, 1H), 7.62 (s, 1H), 7.62 (s, 1H), 7.49 (d, 1H), 7.22 (s, 1H), 7.12 (s, 1H), 6.88 (s, 1H), 3.99 (t, 2H), 3.10 (t, 2H), 2.55 (s, 3H), 0.79 (s, 9H), 0.05 (s, 6H).

MS (m/z): 545 [MH]⁺.

Intermediate 68

### 4-({3-(2-{[(1,1-Dimethylethyl)(dimethyl)silyl]oxy}ethyl)-6-methyl-4-[3-(2-pyridinyl)-1H-pyrazol-1-yl]-2-pyridinyl}amino)-3-(trifluoromethyl)benzonitrile

As in intermediate 9, except that intermediate 65 was used instead of intermediate 8, and 4-amino-3-(trifluoromethyl)benzonitrile was used instead of intermediate 34.

NMR (¹H, CDCl₃): δ 8.64-6.93 (m, 7H), 4.7 (bs, 1H), 4.10 (t, 2H), 2.88 (t, 2H), 2.55 (s, 3H), 0.77 (s, 9H), 0.08 (s, 6H).

MS (m/z): 579 [MH]⁺.

Intermediate 69

### 4-({3-(2-Hydroxyethyl)-6-methyl-4-[3-(2-pyridinyl)-1H-pyrazol-1-yl]-2-pyridinyl}amino)-3-methylbenzonitrile

As in intermediate 21, except that intermediate 66 was used instead of intermediate 9.

NMR (¹H, CDCl₃): δ 8.65 (dd, 1H), 8.15 (m, 2H), 7.95 (d, 1H), 7.75 (m, 2H), 7.42 (m, 2H), 7.26 (m, 1H), 7.15 (d, 1H), 6.80 (s, 1H), 4.27 (t, 2H), 2.84 (t, 2H), 2.51 (s, 3H), 2.30 (s, 3H).

MS (m/z): 411 [MH]⁺.

Intermediate 70

### 3-Chloro-4-({3-(2-hydroxyethyl)-6-methyl-4-[3-(2-pyridinyl)-1H-pyrazol-1-yl]-2-pyridinyl}amino)benzonitrile

As in intermediate 21, except that intermediate 67 was used instead of intermediate 9.

NMR (¹H, CDCl₃): δ 8.65 (d, 1H), 8.55 (d, 1H), 8.46 (s, 1H), 7.96 (d, 1H), 7.77 (m, 2H), 7.64 (s, 1H), 7.50 (d, 1H), 7.26 (d, 1H), 7.17 (d, 1H), 6.85 (s, 1H), 4.25 (t, 2H), 3.72 (bs, 1H), 2.92 (t, 2H), 2.54 (s, 3H).

MS (m/z): 431 [MH]⁺.

Intermediate 71

### 4-({3-(2-Hydroxyethyl)-6-methyl-4-[3-(2-pyridinyl)-1H-pyrazol-1-yl]-2-pyridinyl}amino)-3-(trifluoromethyl)benzonitrile

As in intermediate 21, except that intermediate 68 was used instead of intermediate 9.

NMR (¹H, CDCl₃): δ 8.65 (dd, 1H), 8.53 (bs, 1H), 8.31 (d, 1H), 7.95 (d, 1H), 7.84 (dd, 1H), 7.77-7.68 (m, 3H), 7.25 (m, 1H), 7.17 (d, 1H), 6.80 (s, 1H), 4.18 (m, 2H), 3.41 (bs, 1H), 2.87 (t, 2H), 2.52 (s, 3H).

MS (m/z): 465 [MH]⁺.

Intermediate 72

### 2-(1H-Pyrazol-3-yl)pyridine

The title compound was prepared according to an already published procedure: Pleier, Anna-Katharina; Glas, Holger; Grosche, Manja; Sirsch, Peter; Thiel, Werner R.; Synthesis, 2001, 1, p. 55-62

Intermediate 73

### 2-Methyl-6-(methyloxy)pyridine

To a solution of 6-methyl-pyridin-2-ol (2.5 g, 0.023 mol) in CH₂Cl₂ (10 mL) at r.t., under N₂, were added Ag₂CO₃ (6 g, 1.5 eq.) and MeI (5,6 mL, 4 eq). The solution was stirred at r.t. for 4 days, then Ag₂CO₃ was filtered and washed with CH₂Cl₂, and the organic layer was evaporated to dryness. The crude product was purified by flash chromatography (silica gel, EtOAc/cHex 2:8) to give the title compound (1.9 mg, 67%) as a white solid.

NMR (¹H, CDCl3): δ 7.4 (t, 1H), 6.6 (d,1H), 6.5 (d,1H), 3.8 (s, 3H), 2.4 (s, 3H).

Intermediate 74

### 2-Methyl-6-(methyloxy)-3-nitropyridine

To intermediate 73 (1.95 g, 0.015 mol) at r.t., was added HNO₃ 60% (7 ml). The mixture became reddish brown with generation of heat and NO₂. Conc. H₂SO₄ (7 ml) was then added. The solution was stirred at 80° C. overnight. Into the cooled reaction mixture was poured ice water (70 ml) and the solution was neutralized with CaCO₃. The aqueous solution was extracted with EtOAc (3×50 mL) and the combined organic extracts were dried over anh. Na₂SO₄. The solids were filtered and the solvent was evaporated to dryness to give the title compound (2.25 g, 86%) as a yellow solid.

NMR (¹H, DMSO): δ 8.3 (d,1H); 6.8 (d, 1H); 4 (s, 3H), 2.7 (s, 3H).

Intermediate 75

### 2-Methyl-6-(methyloxy)-3-pyridinamine

To a solution of intermediate 74 (2 g, 0.012 mol) in H₂O/MeOH 1:1 (40 mL), were added NH₄Cl (2.2 g, 3.5 eq.) and Fe powder (2.3 g, 3.5 eq.). The reaction mixture was stirred at 80° C. for 16 hr. Fe was then filtered and washed with MeOH. The MeOH was evaporated, H₂O was added and the aqueous solution was extracted with EtOAc (3×50 mL). The combined organic extracts were dried over anh. Na₂SO₄, the solids were filtered and the solvent evaporated to dryness to give the title compound (1.35 g, 81%) as a brown oil.

NMR (¹H, CDCl₃): δ 6.9 (d,1H), 6.4 (d, ₁H), 3.8 (s, 3H), 2.33 (s, 3H).

Intermediate 76

### {2-Chloro-6-methyl-4-[3-(1,3-thiazol-2-yl)-1H-pyrazol-1-yl]-3-pyridinyl}methyl methanesulfonate

To a solution of intermediate 3 (1 g, 3.26 mmols) in anh. CH₂Cl₂/DMF (15/15 mL), at −15° C., under N₂, were slowly added Et₃N (0.92 mL, 2 eq) and MsCl (0.39 mL, 1.5 eq). The reaction mixture was stirred at −15° C. for 2.5 hr. It was then quenched with water (10 mL) and extracted with CH₂Cl₂ (3×20 mL). The combined organic extracts were washed with sat.aq. NaCl (10 mL) and dried over anh. Na₂SO₄. The solids were filtered and the solvent evaporated. The crude product was purified by flash chromatography (silica gel, cHex/EtOAc 6:4→4:6) to give the title compound as a white solid (1 g, 85%).

NMR (¹H, CDCl₃): δ 7.90 (d, 1H), 7.87 (d, 1H), 7.39 (d, 1H), 7.34 (s, 1H), 7.14 (d, 1H), 5.5 (s, 2H), 3.00 (s, 3H), 2.78 (s, 3H).

MS (m/z): 385 [MH]⁺.

Intermediate 77

### {2-Chloro-6-methyl-4-[3-(1,3-thiazol-2-yl)-1H-pyrazol-1-yl]-3-pyridinyl]acetonitrile

To a solution of intermediate 76 (8 g, 20.79 mmols) in anh. DMF (200 mL), at 0° C., under N₂, was added KCN (1.35 g, 1 eq). The reaction mixture was stirred at r.t. for 6 hr. It was then diluted with Et₂O (500 mL), washed with 1M NaOH (2×200 mL), with sat.aq. NaCl (2×200 mL) and dried over anh. Na₂SO₄. The solids were filtered and the solvent evaporated. The title compound was obtained as a pale brown solid (6.5 g, 96%) and was used in the next step without further purification.

NMR (¹H, CDCl₃): δ 7.92 (d, 1H), 7.91 (d, 1H), 7.41 (d, 1H), 7.31 (s, 1H), 7.18 (d, 1H), 3.99 (s, 2H), 2.78 (s, 3H).

MS (m/z): 316 [MH]⁺.

Intermediate 78

### 2-{2-Chloro-6-methyl-4-[3-(1,3-thiazol-2-yl)-1H-pyrazol-1-yl]-3-pyridinyl}ethyl)amine hydrochloride

To a solution of intermediate 77 (6 g, 19.0 mmols) in anh. THF (6 mL), at r.t., under N₂, was added BH₃.THF 1M/THF (3 eq., 57 mL). The reaction mixture was stirred at reflux for 2 hr. It was then cooled to r.t. and HCl 1M/Et₂O (6 eq with respect to BH₃.THF, exothermic reaction) and MeOH (10 mL) were slowly added. The resulting solution was heated at reflux for 30 min and then concentrated in vacuo. The crude product was dissolved in absolute EtOH and cooled to 0° C. Et₂O was added (60 mL) and a precipitate formed. The solid was filtered and dried to give the title compound as a pale yellow solid (6.7 g, 98%).

NMR (¹H, CDCl₃): δ 8.42 (d, 1H), 7.94 (d, 1H), 7.79 (d, 1H), 7.48 (s, 1H), 7.07(d, 1H), 2.81 (m, 4H), 2.51 (s, 3H), 2.0 (bs, 2H).

MS (m/z): 320.3 [MH]⁺.

Intermediate 79

### 6-Methyl-4-[3-(1,3-thiazol-2-yl)-1H-pyrazol-1-yl]-2,3-dihydro-1H-pyrrolo[2,3-b]pyridine

To a solution of intermediate 78 (1 g, 2.81 mmols) in anh. NMP (15 mL), at r.t., under N₂, were added CuI (107 mg, 0.2 eq), K₂CO₃ (1.17 g, 3 eq) and (1R,2R)-diaminomethylcyclohexane (240 mg, 0.6 eq). The reaction mixture was stirred at 150° C. for 4 hr. It was then diluted with water and extracted with EtOAc (2×20 mL). The combined organic extracts were washed with sat.aq. NaCl and dried over anh. Na₂SO₄. The solids were filtered and the solvent evaporated in vacuo. The crude product was purified by flash chromatography (silica gel CH₂Cl₂/MeOH 98:2). The title compound was obtained as a white solid (400 mg, 50%).

NMR (¹H, CDCl₃): δ 7.98 (d, 1H), 7.89 (d, 1H), 7.35 (d, 1H), 7.06 (d, 1H), 4.65 (bs, 1H), 3.72 (t, 2H), 3.48 (t, 2H), 2.42 (s, 3H)

MS (m/z): 284.3 [MH]⁺

Intermediate 80

### 1-[2-Methyl-4-(methyloxy)phenyl]-2-pyrrolidinone

To a solution of Et₃N (156 mL, 1 eq) and 2-methyl-4-(methyloxy)aniline (150 g, 1.09 mole) in anh. THF (2.4 L), in a 10 L reaction vessel, at 0° C., under N₂, was added dropwise a solution of 4-chlorobutyryl chloride (126 mL, 1 eq) in anh. THF (480 mL). The internal temperature was maintained at circa 10° C. and the reaction mixture was stirred for 1.5 hr. It was cooled down to 0° C. and KOt-Bu 1M/THF (2.64 L, 2.4 eq) was added dropwise over a period of 1.5 hr, keeping the internal temperature <10° C. The reaction mixure was stirred at that temperature for 30 min. Water (1.5 L) was then added slowly (20 min) and the phases were separated. The organic layer was treated with conc. HCl (250 mL) and water (1.26 L) and the phases were separated. The combined aqueous layers were extracted with EtOAc (2.6 L) and the combined organic layers were washed with sat.aq. NaCl (2 L). The solvent was evaporated and the residue purified by flash chromatography (Biotage 150, EtOAc/cHex 8:2) to give the title compound as a pale brown solid (206 g, 92%).

NMR (¹H, CDCl₃): δ 7.05 (d, 1H), 6.79-6.72 (m, 2H), 3.75 (s, 3H), 3.64 (t, 2H), 2.18 (s, 6H).

MS (m/z): 206 [MH]⁺.

Intermediate 81

### Ethyl 3-({1-[2-methyl-4-(methyloxy)phenyl]-2-pyrrolidinylidene}amino)-2-butenoate

To a solution of intermediate 80 (8.3 g, 40.49 mmols) in anh. 1,2-dichloroethane (100 mL), at r.t., under N₂, was added POCl₃ (7.5 mL, 2 eq) dropwise followed by ethyl 3-aminocrotonate (5.17 mL, 1 eq). The reaction mixture was heated at 60° C. for 3.5 hr. It was then cooled down to r.t. and neutralized to pH 7 by the carefull addition of sat.aq. NaHCO₃. The neutralized solution was extracted with CH₂Cl₂ (3×50 mL). The combined organic extracts were washed with sat.aq. NaCl and dried over anh. Na₂SO₄. The solids were filtered and the solvent evaporated. The crude product was used as such in the next step (17.8 g).

MS (m/z): 317 [MH]⁺.

Intermediate 82

### 6-Methyl-1-[2-methyl-4-(methyloxy)phenyl]-1,2,3,7-tetrahydro-4H-pyrrolo[2,3-b]pyridin-4-one

A solution of intermediate 81 (17.8 g, 55 mmols) in anh. DMF (50 mL) was added dropwise to a suspension of NaH 60%/oil (4.5 g, 2 eq) in anh. DMF. The reaction mixture was heated at 100° C. for 8 hr. More NaH 60%/oil (2.25 g, 1 eq) was added and the reaction mixture was heated for an additional 4 hr. It was cooled down to r.t. and carefully poured in sat.aq. NH₄Cl. The aqueous solution was extracted with CH₂Cl₂ (5×50 mL) and the combined organic extracts were dried over anh. Na₂SO₄. The solids were filtered and the solvent was evaporated. The crude compound was purified by flash chromatography (Biotage 75, CH₂Cl₂/MeOH 95:5→80:20). The title compound was obtained as a brown oil (952 mg, 9%, two steps)

NMR (¹H, CDCl₃): δ 7.08 (d, 1H), 6.72-6.68 (m, 2H), 5.87 (s, 1H), 3.73 (s, 3H), 3.73 (t, 2H), 2.99 (t,v2H), 2.21 (s, 3H), 2.13 (s, 3H).

MS (m/z): 271 [MH]⁺.

Intermediate 83

### 6-Methyl-1-[2-methyl-4-(methyloxy)phenyl]-2,3-dihydro-1H-pyrrolo[2,3-b]pyridin-4-yl trifluoromethanesulfonate

To a solution of intermediate 82 (950 mg, 3.52 mmols) in anh. CH₂Cl₂ (10 mL), at −20° C., under N₂, were added pyridine (626 μL, 2.2 eq) and triflic anhydride (651 μL, 1.1 eq). The reaction mixture was stirred at r.t. for 2 hr. It was then poured in sat.aq. NH₄Cl (20 mL) and the phases were separated. The aqueous layer was extracted with CH₂Cl₂ (2×10 mL) and the combined organic extracts were dried over anh. Na₂SO₄. The solids were filtered and the solvent evaporated. The crude product was purified by flash chromatography (silica gel, cHex/EtOAc 9:1) to give the title compound as a white solid (913 mg, 64%).

NMR (¹H, CDCl₃): δ 7.12 (d, 1H), 6.81-6.75 (m, 2H), 6.24 (s, 1H), 3.89 (t, 2H), 3.80 (s, 3H), 3.21 (t, 2H), 2.29 (s, 3H), 2.21 (s, 3H).

MS (m/z): 403 [MH]⁺.

Intermediate 84

### 4-Iodo-6-methyl-1-[2-methyl-4-(methyloxy)phenyl]-2,3-dihydro-1H-pyrrolo[2,3-b]pyridine

To a solution of intermediate 83 (913 mg, 2.27 mmols) in anh. NMP (7 mL), at r.t., under N₂, was added KI (1.13 g, 3 eq) and the reaction mixture was stirred at 150° C. for 18 hr. It was then cooled down to r.t. and diluted in water/sat.aq. NaCl. The aqueous phase was extracted with EtOAc (3×30 mL) and the combined organic extracts were dried over anh. Na₂SO₄. The solids were filtered and the solvent evaporated. The crude product was purified by flash chromatography (silica gel, cHex/EtOAc 9:1) to give the title compound as a clear oil, which solidified upon standing (681 mg, 79%).

NMR (¹H, CDCl₃): δ 7.14 (d, 1H), 6.81-6.74 (m, 2H), 6.70 (s, 1H), 3.84 (t, 2H), 3.81 (s, 3H), 3.03 (t, 2H), 2.22 (s, 6H).

MS (m/z): 381 [MH]⁺.

### Example 1

### Synthesis of Representative Compounds of Structure (IVa)

### Example 1-1

### 6-Methyl-1-[6-(methyloxy)-2-(trifluoromethyl)-3-pyridinyl]-4-[3-(1,3-thiazol-2-yl)-1H-pyrazol-1-yl]-2,3-dihydro-1H-pyrrolo[2,3-b]pyridine

To a solution of intermediate 21 (10 mg, 0.021 mmol) in anh. DMF (1.5 mL) at r.t., were added Et₃N (12 μL, 4 eq) and MsCl (4 μL, 2 eq). The solution was stirred at 56° C. for 4 hr. CH₂Cl₂ (5 mL) was added and the reaction quenched with H₂O (3 mL). The aqueous phase was extracted with CH₂Cl₂ (3×2 mL) and the combined organic extracts dried over anh. Na₂SO₄. The solids were filtered and the solvent evaporated to dryness. The crude product was purified by flash chromatography (silica gel, CH₂Cl₂/MeOH 99:1) to give the title compound (2 mg, 21%) as a white foam.

### Example 1-2

### 6-Methyl-1-[2-methyl-6-(methyloxy)-3-pyridinyl]-4-[3-(1,3-thiazol-2-yl)-1H-pyrazol-1-yl]-2,3-dihydro-1H-pyrrolo[2,3-b]pyridine

To a solution of intermediate 22 (126 mg, 0.030 mmol) in anh. CH₂Cl₂ (10 mL) at r.t., under N₂, were added Et₃N (0.0167 mL, 4 eq), PPh₃ (310 mg, 4 eq) and I₂ (305 mg, 4 eq). The reaction mixture was stirred at r.t. for 2 hr. Water (10 mL) was then added and the solution extracted with EtOAc (15 mL). The organic layer was dried over anh. Na₂SO₄, the solids were filtered and the solvent evaporated in vacuo. The crude compound thus obtained was purified by flash chromatography (silica gel, cHex/EtOAc 6:4) to give 5.9 mg of the title compound as a white solid.

### Example 1-3

### 1-[2,6-Bis(methyloxy)-3-pyridinyl]-6-methyl-4-[3-(1,3-thiazol-2-yl)-1H-pyrazol-1-yl]-2,3-dihydro-1H-pyrrolo[2,3-b]pyridine

To a solution of intermediate 23 (46 mg, 0.105 mmol) in anh. CH₂Cl₂ (2 mL) at r.t., under N₂, were added Et₃N (73 μL, 5 eq), polymer bound PPh₃ (174 mg, 5 eq) and CBr₄ (174 mg, 5 eq). The reaction mixture was stirred at r.t. for 2 hr. Water (10 mL) was then added and the solution extracted with EtOAc (15 mL). The organic layer was dried over anh. Na₂SO₄, the solids were filtered and the solvent evaporated in vacuo. The crude compound thus obtained was purified by flash chromatography (silica gel, cHex/EtOAc 7:3) to give 14 mg of the title compound as a white solid.

### Example 1-4

### N,N,4-Trimethyl-5-{6-methyl-4-[3-(1,3-thiazol-2-yl)-1H-pyrazol-1-yl]-2,3-dihydro-1H-pyrrolo[2,3-b]pyridin-1-yl}-2-pyridinamine

To a solution of intermediate 24 (45 mg, 0.1 mmol) in anh. CH₂Cl₂ (2 mL), at r.t., under N₂, were added I₂ (50.8 mg, 2 eq), PPh₃ (52.4 mg, 2 eq) and Et₃N (27 μL, 2 eq). The reaction mixture was stirred at r.t. for 2.5 hr and evaporated to dryness. The crude product was purified by flash chromatography (silica gel, EtOAc/MeOH 95:5→7:3 EtOAc/NH₃ (0.5 M in MeOH)) to give the title compound as a yellow solid (3 mg, 10%).

All the analytical data are set forth in the following Table 1-1.

| | |
|---|---|
| | (IVa) |
| 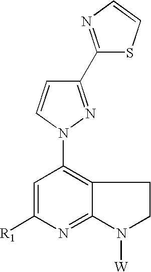 | |

| Cpd. | | | |
|---|---|---|---|
| No. | W | R₁ | Analytical Data |
| 1-1 | 2-trifluoromethyl-4- | CH₃ | NMR (¹H, CDCl₃): δ 7.9 (d, 1H); 7.8 |
| | methoxypyridin-3-yl | | (d, 1H); 7.6 (d, 1H); 7.3 (d, 1H); 7.0 |
| | | | (d, 1H); 6.9 (d, 1H); 6.7 (s, 1H); 3.9 |
| | | | (s, 3H); 3.8 (t, 2H); 3.5 (t, 2H); 2.33 |
| | | | (s, 3H). MS (m/z): 459 [MH]⁺. |
| 1-2 | 2-methyl-4- | CH₃ | NMR (¹H, CDCl₃): δ 7.98 (d, 1H), |
| | methoxy-pyridin- | | 7.88 (d, 1H), 6.98 (d, 1H), 6.83 (d, |
| | 3-yl | | 1H), 7.03 (d, 1H), 6.62 (d, 1H), 6.60 |
| | | | (s, 1H), 3.92 (s, 3H), 3.90 (d, 2H), |
| | | | 3.56 (d, 2H), 2.39 (s, 3H), 2.35 (s, |
| | | | 3H). MS (m/z): 405 [MH]⁺. |
| 1-3 | 2,4- | CH₃ | NMR (¹H, CDCl₃): δ 7.98 (d, 1H); |
| | dimethoxypyridin- | | 7.87 (d, 1H); 7.72 (d, 1H); 7.34 (d, |
| | 3-yl | | 1H); 7.06 (d, 1H); 6.67 (s, 1H); 6.36 |
| | | | (d, 1H); 3.96 (t, 2H); 3.96 (s, 3H); |
| | | | 3.94 (s, 3H); 3.51 (t, 2H); 2.37 (s, |
| | | | 3H)δ. MS (m/z): 421 [MH]⁺. |
| 1-4 | 6-methyl-4- | CH₃ | NMR (¹H, DMSO-d₆): δ 8.57 (d, |
| | dimethyl-amino- | | 1H), 7.95 (s, 1H), 7.92 (d, 1H), 7.78 |
| | pyridin-3-yl | | (d, 1H), 7.04 (d, 1H), 6.86 (s, 1H), |
| | | | 6.55 (s, 1H), 3.88 (t, 2H), 3.49 (t, |
| | | | 2H), 3.16 (s, 3H), 3.14 (s, 3H), 3.01 |
| | | | (s, 6H). IR (, cm⁻¹): |
| | | | MS (m/z): 418 [MH]⁺. |

### Example 2

### Synthesis of Representative Compounds of Structure (Va)

### Example 2-1

### 1-(2-Difluoromethyl-4-methoxy-phenyl)-6-methyl-4-(3-thiazol-2-yl-pyrazol-1-yl)-2,3-dihydro-1H-pyrrolo[2,3-b]pyridine

To a solution of intermediate 25 (42 mg, 0.092 mmol) in anh. CH₂Cl₂ (8 mL) at r.t., under N₂, were added PPh₃ (48 mg, 2 eq) and CBr₄(61 mg, 2 eq). The reaction mixture was stirred at r.t. for 6 hr. Sat.aq. NaHCO₃ (10 mL) was added. The phases were separated and the aqueous layer was extracted with EtOAc (2×20 mL) and the combined organic extracts were dried over anh. Na₂SO₄. The solids were filtered and the solvent was evaporated to dryness. The crude product was purified by flash chromatography (silica gel, cHex/EtOAc 8:2) to give the title compound (19 mg, 50%) as a white solid.

### Example 2-2

### 1-(2-Chloro-4-methoxy-phenyl)-6-methyl-(3-thiazol-2-yl-pyrazol-1-yl)-2,3-dihydro-1H-pyrrolo[2,3-b]pyridine

To a solution of intermediate 26 (25 mg, 0.056 mmol) in anh. DMF (2 mL) at r.t., under N₂, were added Et₃N (30 mL, 4 eq) and MsCl (10 μL, 2 eq). The solution was stirred at 50° C. for 5 hr then CH₂Cl₂ (5 mL) was added and the reaction quenched with H₂O (2 mL). The phases were separated and the aqueous layer was extracted with CH₂Cl₂ (3×3 mL). The combined organic extracts were dried over anh. Na₂SO₄, the solids were filtered and the solvent evaporated under reduced pressure. The crude product was purified by flash chromatography (silica gel, CH₂Cl₂/MeOH 99:1) to give the title compound (8 mg, 33%) as a white foam.

### Example 2-3

### 1-[2,4-Bis(methyloxy)phenyl]-6-methyl-4-[3-(1,3-thiazol-2-yl)-1H-pyrazol-1-yl]-2,3-dihydro-1H-pyrrolo[2,3-b]pyridine

To a suspension of intermediate 27 (26 mg, 0.06 mmol) in anh. CH₂Cl₂ (1 mL), at r.t., under N₂, was added Et₃N (17 μl, 3 eq), PPh₃ (31 mg, 2 eq) and I₂ (3 mg, 2 eq). The reaction mixture was stirred at r.t. for 24 hr and then partitioned between CH₂Cl₂/H₂O. The phases were separated and the organic layer was dried over anh. Na₂SO₄. The solids were filtered and the solvent evaporated. The crude product was purified by flash chromatography (silica gel, 100% CH₂Cl₂→CH₂Cl₂/MeOH 97:3) to give the title compound as a white solid (5 mg, 20%).

### Example 2-4

### 3-Chloro-4-6-methyl-4-3-(1,3-thiazol-2-yl)-1H-pyrazol-1-yl]-2,3-dihydro-1H-pyrrolo[2,3-b]pyridin-1-yl}benzonitrile

To a solution of intermediate 79 (50 mg, 0.176 mmol) in anh. DME (1 mL) were added Pd₂(dba)₃ (16 mg, 0.1 eq), 2-(dicyclohexylphosphino)-2′-methylbiphenyl (19 mg, 0.3 eq), K₃PO₄ (101 mg, 2.7 eq) and 4-amino-3-chlorobenzonitrile (47 mg, 1 eq). The reaction mixture was subjected to microwave irradiation (150W, 100° C., 60 psi) for 20 min. It was then quenched with sat.aq. NH₄Cl and extracted with EtOAc (2×10 mL). The combined organic extracts were washed with sat.aq. NaCl and dried over anh. Na₂SO₄. The solids were filtered and the solvent evaporated. The crude product was purified by flash chromatography (silica gel, cHex/EtOAc 8:2→7:3) to give the title compound as a white solid (16 mg, 22%).

### Example 2-5

### 4-{6-Methyl-4-[3-(1,3-thiazol-2-yl)-1H-pyrazol-1-yl]-2,3-dihydro-1H-pyrrolo[2,3-b]pyridin-1-yl}-3-[(trifluoromethyl)oxy]benzonitrile

To a solution of intermediate 28 (93 mg, 0.191 mmol) in anh. CH₂Cl₂ (3 mL), at r.t., under N₂, were added CBr₄ (317 mg, 5 eq) and PPh₃ (317 mg, 5 eq). The reaction mixture was stirred at r.t. for 2 hr. Water (5 mL) was then added and the solution extracted with CH₂Cl₂ (2×10 mL). The combined organic extracts were dried over anh. Na₂SO₄, the solids were filtered and the solvents evaporated in vacuo. The crude compound thus obtained was purified by flash chromatography (silica gel, cHex/EtOAc 1:1) to give 37 mg of the title compound as a white solid.

### Example 2-6

### 3-Ethyl-4-{6-methyl-4-3-(1,3-thiazol-2-yl)-1H-pyrazol-1-yl]-2,3-dihydro-1H-pyrrolo[2,3-b]pyridin-1-yl}benzonitrile

To a suspension of intermediate 29 (45 mg, 0.105 mmol) in anh. CH₂Cl₂ (2 mL), at r.t., under N₂, was added Et₃N (58 μl, 4 eq) and MsCl (16 μl, 2 eq). The reaction mixture was stirred at r.t. for 24 hr and then partitioned between CH₂Cl₂/H₂O. The phases were separated and the organic layer was dried over anh. Na₂SO₄. The solids were filtered and the solvent evaporated. The crude product was purified by flash chromatography (silica gel, 100% CH₂Cl₂→CH₂Cl₂/MeOH 98:2) to give the title compound as a white solid (2 mg, 5%).

### Example 2-7

### 1-(4-Fluoro-2-methylphenyl)-6-methyl-4-[3-(1,3-thiazol-2-yl)-1H-pyrazol-1-yl]-2,3-dihydro-1H-pyrrolo[2,3-b]pyridine

To a solution of intermediate 30 (7.6 mg, 1 eq) in anh. CH₂Cl₂ (5 mL), at r.t., under N₂, were added Et₃N (9 μl, 3.5 eq) and methanesulfonyl chloride (4.3 μl, 3 eq) and the reaction mixture was stirred at r.t. for 2 hr. It was then partitioned between EtOAc/sat.aq. NaCl. The phases were separated and the organic layer was washed with sat.aq. NaCl (2×10 mL). It was dried over anh. Na₂SO₄, the solids were filtered and the solvent evaporated. The crude product was purified by flash chromatography (silica gel, cHex/EtOAc 7:3) to give the title compound as a yellow solid (0.9 mg, 13%).

### Example 2-8

### 6-Methyl-1-[2-methyl-4-(1H-pyrazol-1-yl)phenyl]-4-3-(1,3-thiazol-2-yl)-1H-pyrazol-1-yl]-2,3-dihydro-1H-pyrrolo[2,3-b]pyridine

To a solution of intermediate 31 (30 mg, 0.065 mmol) in anh. CH₂Cl₂ (2 mL), at r.t., under N₂, were added 12 (66 mg, 4 eq), PPh₃ (68 mg, 4 eq) and Et₃N (36;L, 4 eq). The reaction mixture was stirred at r.t. for 3.5 hr. Sat.aq. NaCl was added and the phases were separated. The aqueous layer was further extracted with CH₂Cl₂ (2×10 mL) and EtOAc (2×10 mL). The combined organic extracts were dried over anh. Na₂SO₄, the solids were filtered and the solvent evaporated. The residue was purified by SCX Column (Eluents: CH₂Cl₂, MeOH and NH₃(0.5 M in MeOH)), then by preparative HPLC (Column: X Terra MS C18 5 um, 100×19 mm/Mobile phase: A: H2O+0.1% TFA; B: CH3CN+0.1% TFA/Gradient: 25% (B) for 6 min, from 25% (B) to 50% (B) in 10 min/Flow rate: 17 ml/min/UV wavelenght range: 200-400 nm/Mass range: 100-900 amu/Ionization: ES+) to give the title compound as a white solid (5 mg, 17%).

All the analytical data are set forth in the following Table 2-1.

| | |
|---|---|
| | (Va) |
| 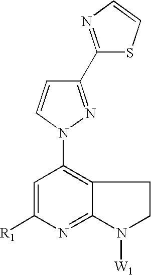 | |

| Cpd. | | | |
|---|---|---|---|
| No. | W1 | R₁ | Analytical Data |
| 2-1 | 2-difluoromethyl-4- | CH₃ | NMR (¹H, CDCl₃): δ 8.00 (d, 1H), |
| | methoxyphenyl | | 7.89 (d, 1H), 7.36 (d, 1H), 7.24 (m, |
| | | | 2H), 7.08 (m, 2H), 6.88 (t, 1H; |
| | | | J_{(H-F)} =55.8 Hz), 6.71 (s, 1H), 3.95 (t, |
| | | | 2H), 3.87 (s, 3H), 3.56 (t, 2H), 2.36 |
| | | | (s, 3H). MS (m/z): 440 [MH]⁺. |
| 2-2 | 2-chloro-4-methoxy- | CH₃ | NMR (¹H, CDCl₃): δ 7.97 (bs, 1H), |
| | phenyl | | 7.86 (bs, 1H), 7.36-7.32 (m, 2H), |
| | | | 7.01 (d, 2H), 6.85 (m, 1H), 6.66 (s, |
| | | | 1H), 3.96 (t, 2H), 3.79 (s, 3H), 3.54 |
| | | | (t, 2H), 2.35 (s, 3H). MS (m/z): |
| | | | 424 [MH]⁺ |
| 2-3 | 2,4- | CH₃ | NMR (¹H, CDCl₃): δ 7.97 (d, 1H), |
| | dimethoxyphenyl | | 7.86 (d, 1H), 7.34 (d, 1H), 7.04 (d, |
| | | | 1H), 6.64 (s, 1H), 6.54 (d, 1H), 6.51 |
| | | | (d, 1H), 3.82 (s, 3H), 3.79 (s, 3H), |
| | | | 3.53-3.46 (m, 4H), 2.36 (s, 3H). |
| | | | MS (m/z): 421 [MH]⁺. |
| 2-4 | 2-chloro-4-cyano- | CH₃ | NMR (¹H, CDCl₃): δ 8.02 (d, 1H), |
| | phenyl | | 7.90 (d, 1H), 7.74 (d, 1H), 7.73 (d, |
| | | | 1H), 7.56 (dd, 1H), 7.37 (d, 1H), |
| | | | 7.09 (d, 1H), 6.83 (s, 1H), 4.16 (t, |
| | | | 2H), 3.62 (t, 2H), 2.43 (s, 3H). |
| | | | MS (m/z): 419 [MH]⁺. |
| 2-5 | 2- | CH₃ | NMR (¹H, CDCl₃): δ 8.66 (d, 1H); |
| | trifluoromethyloxy- | | 8.03 (s, 1H); 7.96 (d, 1H); 7.94 (d, |
| | 4-cyanophenyl | | 1H); 7.88 (dd, 1H); 7.81 (d, 1H); |
| | | | 7.21 (s, 1H); 7.10 (d, 1H); 4.16 (t, |
| | | | 2H); 3.56 (t, 2H); 2.35 (s, 3H). |
| | | | MS (m/z): 469 [MH]⁺. |
| 2-6 | 2-ethyl-4- | CH₃ | NMR (¹H, DMSO): δ 9.17 (s, 1H), |
| | cyanophenyl | | 8.32 (d, 1H), 7.98 (d, 1H), 7.92 (s, |
| | | | 1H), 7.75 (s, 1H), 7.60 (s, 1H), 7.54 |
| | | | (d, 1H), 7.03 (s, 2H), 7.96 (s, 1H), |
| | | | 4.01 (t, 2H), 3.23 (t, 2H), 2.64 (q, |
| | | | 2H), 2.41 (s, 3H), 1.40 MS (m/z): |
| | | | 414 [MH]⁺. |
| 2-7 | 2-methyl-4- | CH₃ | NMR (¹H, CDCl₃): δ 7.98 (d, 1H), |
| | fluorophenyl | | 7.88 (d, 1H), 7.36 (d, 1H), 7.25 (m, |
| | | | 1H), 7.06 (d, 1H), 7.02/6.9 (m, 2H), |
| | | | 6.57 (s, 1H), 3.93 (t, 2H), 3.56 (t, |
| | | | 2H), 2.36 (s, 3H), 2.27 (s, 3H). |
| | | | MS (m/z): 392 [MH]⁺. |
| 2-8 | 2-methyl-4(1- | CH₃ | NMR (¹H, CDCl₃): δ 7.99 (d, 1H), |
| | pyrazolyl)phenyl | | 7.89 (d, 1H), 7.87 (d, 1H), 7.70 (d, |
| | | | 1H), 7.63 (d, 1H), 7.52 (dd, 1H), |
| | | | 7.35 (d, 1H), 7.34 (d, 1H), 7.06 (d, |
| | | | 1H), 6.66 (s, 1H), 6.44 (t, 1H), 3.98 |
| | | | (t, 2H), 3.57 (t, 2H), 2.36 (s, 3H), |
| | | | 2.33 (s, 3H). MS (m/z): 440 [MH]⁺. |

### Example 3

### Synthesis of Representative Compounds of Structure (VIa)

### Example 3-1

### 6-Methyl-1-[4-nitro-2-(trifluoromethyl)phenyl]-4-[3-(1,3-thiazol-2-yl)-1H-pyrazol-1-yl]-2,3-dihydro-1H-pyrrolo[2,3-b]pyridine

To a solution of intermediate 32 (17 mg, 0.034 mmol) in anh. CH₂Cl₂ (5 mL) at r.t., under N₂, were added Et₃N (0.019 ml), PPh₃ (17.8 mg) and I₂ (26.1 mg). The reaction mixture was stirred at r.t. for 1 hr. Water (10 mL) was then added and the solution extracted with EtOAc (15 mL). The organic layer was dried over anh. Na₂SO₄, the solids were filtered and the solvents evaporated in vacuo. The crude compound thus obtained was purified by flash chromatography (silica gel, cHex/EtOAc 6:4) to give 5.9 mg of the title compound as a yellow solid.

All the analytical data are set forth in the following Table 3-1.

| | |
|---|---|
| | (VIa) |
| 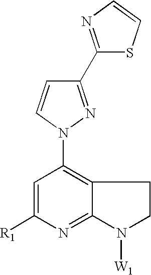 | |

| Cpd. | | | | |
|---|---|---|---|---|
| No. | W1 | R₁ | R₄ | Analytical Data |
| 3-1 | 2-trifluoromethyl-4- | CH₃ | H | NMR (¹H, CDCl₃): δ 8.63 (d, 1H), 8.41 |
| | nitrophenyl | | | (dd, 1H), 8.03 (d, 1H), 7.90 (d, 1H), 7.76 |
| | | | | (d, 1H), 7.37 (d, 1H), 7.10 (d, 1H), 6.85 |
| | | | | (s, 1H), 4.08 (t, 2H), 3.65 (t, 2H), 2.40 |
| | | | | (s, 3H). MS (m/z): 473 [MH]⁺. |

### Example 4

### Synthesis of Representative Compounds of structure (VIIa)

### Example 4-1

### 4-[4-(1′H-1,3′-Bipyrazol-1′-yl)-6-methyl-2,3-dihydro-1H-pyrrolo[2,3-b]pyridin-1-yl]-3-methyl-benzonitrile

To a solution of intermediate 54 (20 mg, 0.049 mmol) in anh. CH₂Cl₂ (2.5 mL), at r.t., under N₂, were added triphenylphosphine (78 mg, 5 eq) and tetrabromomethane (83 mg 5 eq) and the reaction mixture was stirred at r.t. for 60 min. It was partitioned between CH₂Cl₂/sat.aq. NaHCO₃. The phases were separated and the organic layer was washed with sat.aq. NaCl (2×10 mL). It was dried over anh. Na₂SO₄, the solids were filtered and the solvent evaporated. The crude product was purified by flash chromatography (silica gel, 7:3 cHex/EtOAc) to give the title compound as a white solid (9 mg, 48%).

### Example 4-2

### 4-[4-(1′H-1,3′-Bipyrazol-1′-yl)-6-methyl-2,3-dihydro-1H-pyrrolo[2,3-b]pyridin-1-yl]-3-(trifluoro-methyl)benzonitrile

To a solution of intermediate 55 (85 mg, 0.187 mmol) in anh. CH₂Cl₂ (5.5 mL), at r.t., under N₂, were added triphenylphosphine (98 mg, 2.5 eq) and tetrabromomethane (124 mg 2.5 eq) and the reaction mixture was stirred at r.t. for 60 min. It was partitioned between CH₂Cl₂/sat.aq. NaHCO₃. The phases were separated and the organic layer was washed with sat.aq. NaCl (2×10 mL). It was dried over anh. Na₂SO₄, the solids were filtered and the solvent evaporated. The crude product was purified by flash chromatography (silica gel, 7:3 cHex/EtOAc) to give the title compound as a solid (42 mg, 51%).

### Example 4-3

### [4-{4-(1′H-1,3′-Bipyrazol-1′-yl)-6-methyl-2,3-dihydro-1H-pyrrolo[2,3-b]pyridin-1-yl]-3-chloro-benzonitrile

To a solution of intermediate 56 (39 mg, 0.094 mmol) in anh. CH₂Cl₂ (5.5 mL), at r.t., under N₂, were added triphenylphosphine (49 mg, 2.5 eq) and tetrabromomethane (62 mg 2.5 eq) and the reaction mixture was stirred at r.t. for 60 min. It was partitioned between CH₂Cl₂/sat.aq. NaHCO₃. The phases were separated and the organic layer was washed with sat.aq. NaCl (2×10 mL). It was dried over anh. Na₂SO₄, the solids were filtered and the solvent evaporated. The crude product was purified by flash chromatography (silica gel, 7:3 cHex/EtOAc) to give the title compound as a solid (14 mg, 35%).

### Example 4-4

### 1′-[6-Methyl-1-[2-methyl-4-(methyloxy)phenyl]-2,3-dihydro-1H-pyrrolo[2,3-b]pyridin-4-yl}-1′H-1,3′-bipyrazole

To a solution of intermediate 84 (200 mg, 0.53 mmol) in anh. NMP (1 mL), at r.t., under N₂, were added 3-bromo-1H-pyrazole (77 mg, 1 eq.), CuI (101 mg, 1 eq), K₂CO₃ (154 mg, 2.1 eq) and (1R,2R)-diaminomethylcyclohexane (23 mg, 0.3 eq.). The reaction mixture was heated at 150° C. for 3.5 h. It was cooled down to r.t., poured into water/EtOAc and the phases were separated. The aqueous layer was further extracted with EtOAc (2×10 mL). The combined organic extracts were dried over anh. Na₂SO₄, the solids were filtered and the solvent evaporated. The residue was purified by flash chromatography (silica gel, 95:5 cHex/EtOAc, two purifications) to give the title compound as a white solid (7.5 mg, 4%).

All the analytical data are set forth in the following Table 4-1.

| | |
|---|---|
| | (VIIa) |
| 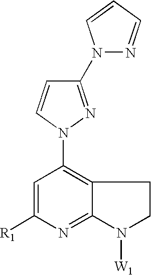 | |

| Cpd. | | | |
|---|---|---|---|
| No. | W1 | R₁ | Analytical Data |
| 4-1 | 2-methyl-4-cyano- | CH₃ | NMR (¹H, CDCl₃): δ 8.2 (d, 1H), |
| | phenyl | | 8.0 (d, 1H), 7.7 (d, 1H), 7.6 (d, 1H), |
| | | | 7.5 (dd, 1H), 7.4 (dd, 1H), 6.8 (d, |
| | | | 1H), 6.7 (d, 1H), 6.4 (t, 1H), 4.0 (t, |
| | | | 2H), 4.0 (t, 2H), 3.6 (t, 2H), 2.41, |
| | | | 2.35 (s + s, 6H). MS (m/z): |
| | | | 382 [MH]⁺. |
| 4-2 | 2-trifluoromethyl-4- | CH₃ | NMR (¹H, CDCl₃): δ 8.2 (d, 1H), |
| | cyanophenyl | | 8.0 (d + d, 2H), 7.8 (dd, 1H), 7.7 |
| | | | (d, 1H), 7.6 (dd, 1H), 6.8 (m, 2H), |
| | | | 6.4 (d, 1H), 4.0 (t, 2H), 3.6 (t, 2H), |
| | | | 2.41 (s, 3H). MS (m/z): 436 [MH]⁺. |
| 4-3 | 2-chloro-4-cyano- | CH₃ | NMR (¹H, CDCl₃): δ 8.2 (d, 1H), |
| | phenyl | | 8.0 (d, 1H), 7.8 (m, 3H), 7.6 (d, 1H), |
| | | | 6.8 (m, 2H), 6.4 (d, 1H), 4.2 (t, 2H), |
| | | | 3.6 (t, 2H), 2.40 (s, 3H). |
| | | | MS m/z: 402 [MH]⁺. |
| 4-4 | 2-methyl-4- | CH₃ | NMR (¹H, Acetone-d₆): δ 8.15 (d, |
| | methoxy-phenyl | | 1H), 7.86 (d, 1H), 7.66 (d, 1H), 7.12 |
| | | | (d, 1H), 6.76 (d, 1H), 6.72 (dd, 1H), |
| | | | 6.54 (s, 1H), 6.39 (dd, 1H), 3.84 (t, |
| | | | 2H), 3.74 (s, 3H), 3.44 (t, 2H), 2.28 |
| | | | (s, 3H), 2.18 (s, 3H). |
| | | | MS m/z: 387 [MH]⁺. |

### Example 5

### Synthesis of Representative Compounds of Structure (VIIIa)

### Example 5-1

### 3-Methyl-4-{6-methyl-4-[3-(2-pyridinyl)-1H-pyrazol-1-yl]-2,3-dihydro-1H-pyrrolo[2,3-b]pyridin-1-yl}benzonitrile

To a solution of intermediate 69 (24 mg, 0.058 mmol) in anh. CH₂Cl₂ (2 mL), at r.t., under N₂, were added PPh₃ (30 mg, 2 eq) and CBr₄ (29 mg, 1.5 eq) and the reaction mixture was stirred at r.t. for 18 hr. It was partitioned between CH₂Cl₂/sat.aq. NaHCO₃. The phases were separated and the organic layer was washed with sat.aq. NaCl (2×10 mL). It was dried over anh. Na₂SO₄, the solids were filtered and the solvent evaporated. The crude product was purified by flash chromatography (silica gel, cHex/EtOAc 7:3) to give the title compound as a solid (8 mg, 35%).

### Example 5-2

### 3-Chloro-4-{6-methyl-4-[3-(2-pyridinyl)-1H-pyrazol-1-yl]-2,3-dihydro-1H-pyrrolo[2,3-b]pyridin-1-yl]benzonitrile

To a solution of intermediate 70 (39 mg, 0.094 mmol) in anh. CH₂Cl₂ (5 mL), at r.t., under N₂, were added imidazole (18 mg, 3 eq), triphenylphosphine (47 mg, 2 eq) and iodine (69.3 mg, 3 eq) and the reaction mixture was stirred at r.t. for 1 hr. It was then partitioned between EtOAc/sat.aq. NaCl. The phases were separated and the organic layer was washed with sat.aq. NaCl (2×10 mL). It was dried over anh. Na₂SO₄, the solids were filtered and the solvent evaporated. The crude product was purified by flash chromatography (silica gel, 1:1 cHex/EtOAc) to give the title compound as a colorless oil (4.5 mg, 12%).

### Example 5-3

### 4-{6-Methyl-4-[3-(2-pyridinyl)-1H-pyrazol-1-yl]-2,3-dihydro-1H-pyrrolo[2,3-b]pyridin-1-yl}-3-(trifluoromethyl)benzonitrile

To a solution of intermediate 71 (44 mg, 0.095 mmol) in anh. CH₂Cl₂ (3 mL), at r.t., under N₂, were added PPh₃ (50 mg, 2 eq) and CBr₄ (47 mg, 1.5 eq) and the reaction mixture was stirred at r.t. for 18 hr. It was partitioned between CH₂Cl₂/sat.aq. NaHCO₃. The phases were separated and the organic layer was washed with sat.aq. NaCl (2×10 mL). It was dried over anh. Na₂SO₄, the solids were filtered and the solvent evaporated. The crude product was purified by flash chromatography (silica gel, cHex/EtOAc 1:1) to give the title compound as a solid (8 mg, 20%).

All the analytical data are set forth in the following Table 5-1.

| | |
|---|---|
| | (VIII |
| 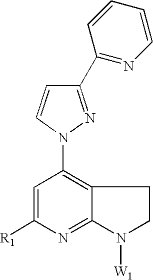 | |

| Cpd. | | | |
|---|---|---|---|
| No. | W1 | R₁ | Analytical Data |
| 5-1 | 2-methyl-4-cyano- | CH₃ | NMR (¹H, CDCl₃): δ 8.65 (dd, 1H), |
| | phenyl | | 8.10 (d, 1H), 8.00 (d, 1H), 7.75 (dt, |
| | | | 1H), 7.55-7.48 (m, 2H), 7.39 (d, |
| | | | 1H), 7.26 (m, 1H), 7.15 (d, 1H), 6.78 |
| | | | (s, 1H), 4.00 (t, 2H), 3.60 (t, 2H), |
| | | | 2.37 (s, 3H), 2.31 (s, 3H). MS (m/z): |
| | | | 393 [MH]⁺. |
| 5-2 | 2-chloro-4-cyano- | CH₃ | NMR (¹H, CDCl₃): δ 8.67 (d, 1H), |
| | phenyl | | 8.11 (d, 1H), 8.03 (d, 1H), 7.75 (m, |
| | | | 2H), 7.75 (m, 1H), 7.55 (dd, 1H), |
| | | | 7.26 (m, 1H), 7.2 (d, 1H), 6.88 (s, |
| | | | 1H), 4.16 (t, 2H), 3.65 (t, 2H), 2.43 |
| | | | (s, 3H). MS (m/z): 413 [MH]⁺. |
| 5-3 | 2-trifluoromethyl- | CH₃ | NMR (¹H, CDCl₃): δ 8.66 (dd, 1H), |
| | 4-cyano-phenyl | | 8.10 (d, 1H), 8.02 (d, 1H), 8.00 (d, |
| | | | 1H), 7.84 (dd, 1H), 7.76 (t, 1H), 7.69 |
| | | | (d, 1H), 7.27 (t, 1H), 7.18 (d, 1H), |
| | | | 6.87 (s, 1H), 4.02 (t, 2H), 3.64 (t, |
| | | | 2H), 2.38 (s, 3H). MS (m/z): |
| | | | 447 [MH]⁺. |

### Example 6

### CRF Binding Activity

CRF binding affinity has been determined in vitro by the compounds' ability to displace ¹²⁵I-oCRF and ¹²⁵I-Sauvagine for CRF1 and CRF2 SPA, respectively, from recombinant human CRF receptors expressed in Chinese Hamster Ovary (CHO) cell membranes. For membrane preparation, CHO cells from confluent T-flasks were collected in SPA buffer (HEPES/KOH 50 mM, EDTA 2 mM; MgCl₂ 10 mM, pH 7.4.) in 50 mL centrifuge tubes, homogenized with a Polytron and centrifuged (50'000 g for 5 min at 4° C.: Beckman centrifuge with JA20 rotor). The pellet was resuspended, homogenized and centrifuged as before.

The SPA experiment has been carried out in Optiplate by the addition of 100 μL the reagent mixture to 1 μL of compound dilution (100% DMSO solution) per well. The assay mixture was prepared by mixing SPA buffer, WGA SPA beads (2.5 mg/mL), BSA (1 mg/mL) and membranes (50 and 5 μg of protein/mL for CRF1 and CRF2 respectively) and 50 pM of radioligand.

The plate was incubated overnight (>18 hrs) at room temperature and read with the Packard Topcount with a WGA-SPA ¹²⁵I counting protocol.

### Example 7

### CRF Functional Assay

Compounds of the invention were characterised in a functional assay for the determination of their inhibitory effect. Human CRF-CHO cells were stimulated with CRF and the receptor activation was evaluated by measuring the accumulation of CAMP.

CHO cells from a confluent T-flask were resuspended with culture medium without G418 and dispensed in a 96-well plate, 25'000c/well, 100 μL/well and incubated overnight. After the incubation the medium was replaced with 100 μL of CAMP IBMX buffer warmed at 37° C. (5 mM KCl, 5 mM NaHCO₃, 154 mM NaCl, 5 mM HEPES, 2.3 mM CaCl₂, 1 mM MgCl₂; 1 g/L glucose, pH 7.4 additioned by 1 mg/mL BSA and 1 mM IBMX) and 1 μL of antagonist dilution in neat DMSO. After 10 additional minutes of incubation at 37° C. in a plate incubator without CO₂, 1 μL of agonist dilution in neat DMSO was added. As before, the plate was incubated for 10 minutes and then CAMP cellular content was measured by using the Amersham RPA 538 kit.

All publications, including but not limited to patents and patent applications, cited in this specification are herein incorporated by reference as if each individual publication were specifically and individually indicated to be incorporated by reference herein as though fully set forth.

It is to be understood that the present invention covers all combinations of particular and preferred groups described herein above.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. Compounds of formula (I) including stereoisomers, prodrugs and pharmaceutically acceptable salts or solvates thereof 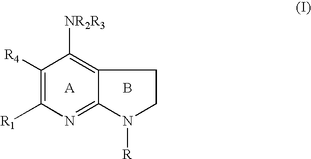
wherein
R is aryl or heteroaryl, each of which may be substituted by 1 or more Z groups;
R1 is hydrogen, C3-C7 cycloalkyl, C1-C6 alkyl, C1-C6 alkoxy, C1-C6 thioalkyl, C2-C6 alkenyl, C2-C6 alkynyl, halo C1-C6 alkyl, halo C1-C6 alkoxy, halogen, NR6R7 or cyano;
R2 and R3 together with N form a 5-6 membered aromatic heterocycle, which is substituted by at least one group R8 and may be further substituted by 1 to 3 R9 groups;
R4 hydrogen, C1-C6 alkyl, halogen or halo C1-C6 alkyl;
R5 is a C1-C4 alkyl, —OR6 or —NR6R7;
R6 is hydrogen or C1-C6 alkyl;
R7 is hydrogen or C1-C6 alkyl;
R8 is a 5-6 membered aromatic heterocycle, which may be substituted by 1 to 4 R10 groups;
R9 is hydrogen, C3-C7 cycloalkyl, C3-C7 cycloalkenyl, C1-C6 alkyl, C1-C6 alkoxy, halo C1-C6 alkyl, halo C1-C6 alkoxy, hydroxy, halogen, nitro, cyano, —C(O)R5, C(O)NR6R7, phenyl which may be substituted by 1 to 4 R10 groups;
R10 is C1-C6 alkyl, halo C1-C2 alkyl, halogen, nitro, hydroxy, halo C1-C6 alkoxy, C1-C6 alkoxy, or cyano;
Z is selected in a group consisting from halogen, C1-C6 alkyl, C1-C6 alkoxy, halo C1-C6 alkyl, C2-C6 alkenyl, C2-C6 alkynyl, halo C1-C6 alkoxy, —C(O)R5, —NR6R7, nitro, cyano, and a group R8;
and when R4 is hydrogen, R2 and R3 together with N form a pyrazolyl group substituted with at least one R8 corresponding to a thiazolyl group, and R corresponds to a phenyl group, and the substituent Z is at least one nitro group,
then in the compounds of formula (I) the nitro group is not present in the ortho position with respect to the nitrogen atom present in the 5-membered ring, named as B;
and the compounds of formula (I) don't include the following:
1-(2,4-bis-trifluoromethyl-phenyl)-6-methyl-4-(3-thiazol-2-yl-pyrazol-1-yl)-2,3-dihydro-1H-pyrrolo[2,3-b]pyridine;
3-methyl-4-[6-methyl-4-(3-thiazol-2-yl-pyrazol-1-yl)-2,3-dihydro-1H-pyrrolo[2,3-b]pyridin-1-yl]-benzonitrile;
4-[6-methyl-4-(3-thiazol-2-yl-pyrazol-1-yl)-2,3-dihydro-1H-pyrrolo[2,3-b]pyridin-1-yl]-3-trifluoromethyl-benzonitrile;
6-methyl-1-(2-methyl-4-trifluoromethoxy-phenyl)-4-(3-thiazol-2-yl-pyrazol-1-yl)-2,3-dihydro-1H-pyrrolo[2,3-b]pyridine;
1-(4-methoxy-2-methyl-phenyl)-6-methyl-4-(3-thiazol-2-yl-pyrazol-1-yl)-2,3-dihydro-1H-pyrrolo[2,3-b]pyridine
1-(2,4-bis-trifluoromethyl-phenyl)-6-methyl-4-(3-pyridin-2-yl-pyrazol-1-yl)-2,3-dihydro-1H-pyrrolo[2,3-b]pyridine
4-[1,3′]bipyrazolyl-1′-yl-1-(2,4-bis-trifluoromethyl-phenyl)-6-methyl-2,3-dihydro-1H-pyrrolo[2,3-b]pyridine.

2. Compounds of formula (II), according to claim 1, in which R4 is hydrogen and R, R1, R2, and R3 are defined as in claim 1.

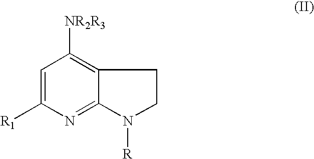

3. Compounds of formula (III), according to claim 1, 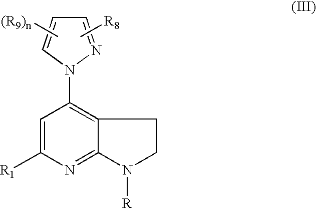
in which
n is an integer from 1 to 2; and
R, R1, and R8 and R9 are defined as in claim 1.

4. Compounds of formula (IV), according to claim 1, 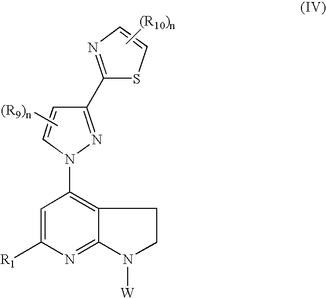
in which:
R8 is a thiazolyl derivative;
n is an integer from 1 to 2;
R corresponds to W and
W is a pyridine derivative, which may be substituted by 1 to 4 Z groups, as defined above; and
R1, R9 and R10 are defined as in claim 1.

5. Compounds of formula (IVa), according to claim 4, 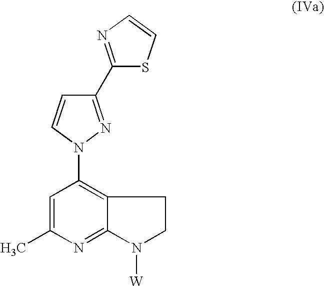
in which W is defined as in claim 4.

6. Compounds of formula (V), according to claim 1, 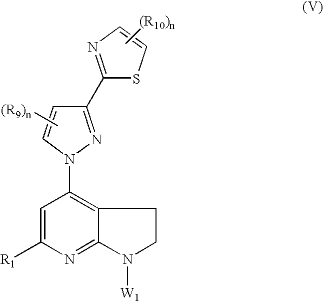
in which:
R8 is a thiazolyl derivative;
n is an integer from 1 to 2;
R corresponds to W1 and
W1 is a phenyl derivative substituted by 2 to 4 Z1 groups;
Z1 is selected in a group consisting from halogen, C1-C6 alkyl, C1-C6 alkoxy, halo C1-C6 alkyl, C2-C6 alkenyl, C2-C6 alkynyl, halo C1-C6 alkoxy, —C(O)R5, —NR6R7, cyano, and a group R8;
and R1, R5, R6, R7, R8, R9 and R10 are defined as in claim 1.

7. Compounds of formula (Va), according to claim 6, 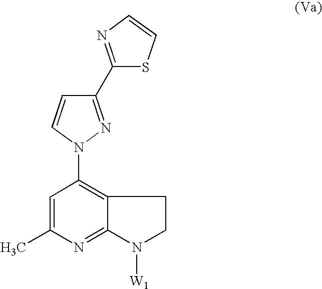
in which W1 is defined as in claim 6.

8. Compounds of formula (VI), according to claim 1, 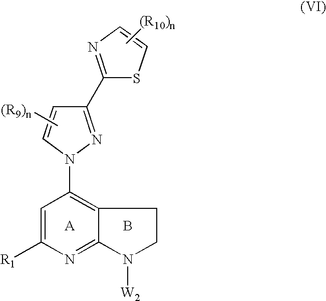
in which:
R8 is a thiazolyl derivative;
n is an integer from 1 to 2;
R corresponds to W2 and
W2 is a phenyl derivative, which may be substituted by 2 to 4 Z groups as defined in claim 1 provided that at least one Z group is nitro and further provided that the nitro group is not in the ortho position with respect to the nitrogen atom of the 5-membered ring named as B; and
R1, R9 and R10 are defined as in claim 1.

9. Compounds of formula (VIa), according to claim 8, 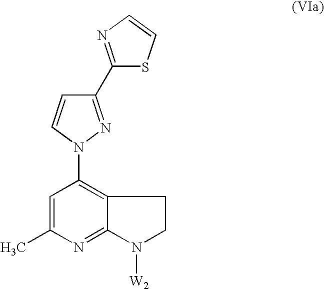
in which W2 is defined as in claim 8.

10. Compounds of formula (VII), according to claim 1, 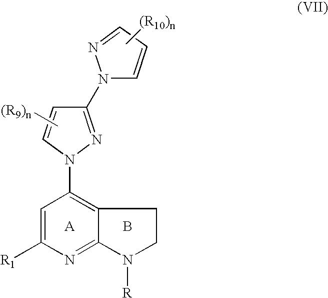
in which
R8 is a pyrazolyl derivative;
n is an integer from 1 to 2;
R, R9 and R10 are defined as in claim 1.

11. Compounds of formula (VIIa), according to claim 1, 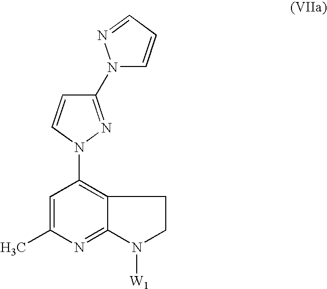
in which
W1 is a phenyl derivative substituted by 2 to 4 Z1 groups;
Z1 is selected in a group consisting from halogen, C1-C6 alkyl, C1-C6 alkoxy, halo C1-C6 alkyl, C2-C6 alkenyl, C2-C6 alkynyl, halo C1-C6 alkoxy, —C(O)R5, —NR6R7, cyano, and a group R8.

12. Compounds of formula (VIII), according to claim 1, 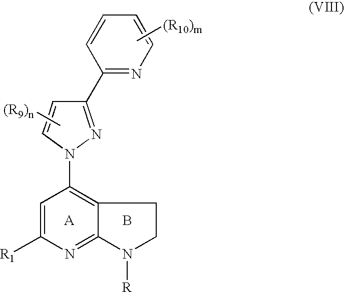
in which
R8 is a pyridine derivative;
n is an integer from 1 to 2;
m is an integer from 1 to 3;
R, R9 and R10 are defined as in claim 1.

13. Compounds of formula (VIIIa), according to claim 1, 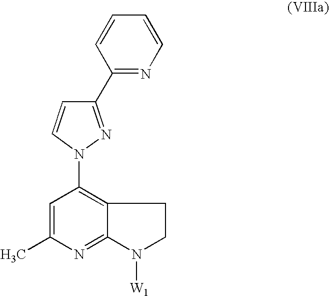
in which
W1 is a phenyl derivative substituted by 2 to 4 Z1 groups:
Z1 is selected in a group consisting from halogen, C1-C6 alkyl, C1-C6 alkoxy, halo C1-C6 alkyl, C2-C6 alkenyl, C2-C6 alkynyl, halo C1-C6 alkoxy, —C(O)R5, —NR6R7, cyano, and a group R8.

14. Compounds according to claim 1 selected in the group consisting from:
6-methyl-1-[6-(methyloxy)-2-(trifluoromethyl)-3-pyridinyl]-4-[3-(1,3-thiazol-2-yl)-1H-pyrazol-1-yl]-2,3-dihydro-1H-pyrrolo[2,3-b]pyridine;
6-methyl-1-[2-methyl-6-(methyloxy)-3-pyridinyl]-4-[3-(1,3-thiazol-2-yl)-1H-pyrazol-1-yl]-2,3-dihydro-1H-pyrrolo[2,3-b]pyridine;
1-[2,6-bis(methyloxy)-3-pyridinyl]-6-methyl-4-[3-(1,3-thiazol-2-yl)-1H-pyrazol-1-yl]-2,3-dihydro-1H-pyrrolo[2,3-b]pyridine;
N,N,4-trimethyl-5-{6-methyl-4-[3-(1,3-thiazol-2-yl)-1H-pyrazol-1-yl]-2,3-dihydro-1H-pyrrolo[2,3-b]pyridin-1-yl}-2-pyridinamine;
1-(2-difluoromethyl-4-methoxy-phenyl)-6-methyl-4-(3-thiazol-2-yl-pyrazol-1-yl)-2,3-dihydro-1H-pyrrolo[2,3-b]pyridine;
1-(2-chloro-4-methoxy-phenyl)-6-methyl-4-(3-thiazol-2-yl-pyrazol-1-yl)-2,3-dihydro-1H-pyrrolo[2,3-b]pyridine;
1-[2,4-bis(methyloxy)phenyl]-6-methyl-4-[3-(1,3-thiazol-2-yl)-1H-pyrazol-1-yl]-2,3-dihydro-1H-pyrrolo[2,3-b]pyridine;
3-chloro-4-{6-methyl-4-[3-(1,3-thiazol-2-yl)-1H-pyrazol-1-yl]-2,3-dihydro-1H-pyrrolo[2,3-b]pyridin-1-yl}benzonitrile;
4-{6-methyl-4-[3-(1,3-thiazol-2-yl)-1H-pyrazol-1-yl]-2,3-dihydro-1H-pyrrolo[2,3-b]pyridin-1-yl}-3-[(trifluoromethyl)oxy]benzonitrile;
3-ethyl-4-6-methyl-4-[3-(1,3-thiazol-2-yl)-1H-pyrazol-1-yl]-2,3-dihydro-1H-pyrrolo[2,3-b]pyridin-1-yl}benzonitrile;
1-(4-fluoro-2-methylphenyl)-6-methyl-4-[3-(1,3-thiazol-2-yl)-1H-pyrazol-1-yl]-2,3-dihydro-1H-pyrrolo[2,3-b]pyridine;
6-methyl-1-[2-methyl-4-(1H-pyrazol-1-yl)phenyl]-4-[3-(1,3-thiazol-2-yl)-1H-pyrazol-1-yl]-2,3-dihydro-1H-pyrrolo[2,3-b]pyridine;
6-methyl-1-[4-nitro-2-(trifluoromethyl)phenyl]-4-[3-(1,3-thiazol-2-yl)-1H-pyrazol-1-yl]-2,3-dihydro-1H-pyrrolo[2,3-b]pyridine;
4-[4-(1H-1,3′-bipyrazol-1′-yl)-6-methyl-2,3-dihydro-1H-pyrrolo[2,3-b]pyridin-1-yl]-3-methylbenzonitrile;
4-[4-(1H-1,3′-bipyrazol-1′-yl)-6-methyl-2,3-dihydro-1H-pyrrolo[2,3-b]pyridin-1-yl]-3-(trifluoromethyl)benzonitrile;
4-[4-(1H-1,3′-bipyrazol-1′-yl)-6-methyl-2,3-dihydro-1H-pyrrolo[2,3-b]pyridin-1-yl]-3-chlorobenzonitrile;
1′-{6-methyl-1-[2-methyl-4-(methyloxy)phenyl]-2,3-dihydro-1H-pyrrolo[2,3-b]pyridin-4-yl}-1′H-1,3′-bipyrazole;
3-methyl-4-{6-methyl-4-[3-(2-pyridinyl)-1H-pyrazol-1-yl]-2,3-dihydro-1H-pyrrolo[2,3-b]pyridin-1-yl} benzonitrile;
3-chloro-4-{6-methyl-4-[3-(2-pyridinyl)-1H-pyrazol-1-yl]-2,3-dihydro-1H-pyrrolo[2,3-b]pyridin-1-yl}benzonitrile;
4-{6-methyl-4-[3-(2-pyridinyl)-1H-pyrazol-1-yl]-2,3-dihydro-1H-pyrrolo[2,3-b]pyridin-1-yl}-3-(trifluoromethyl)benzonitrile.

15. A process for the preparation of a compound of formula (I) as claimed in claim 1, which comprises the following steps: 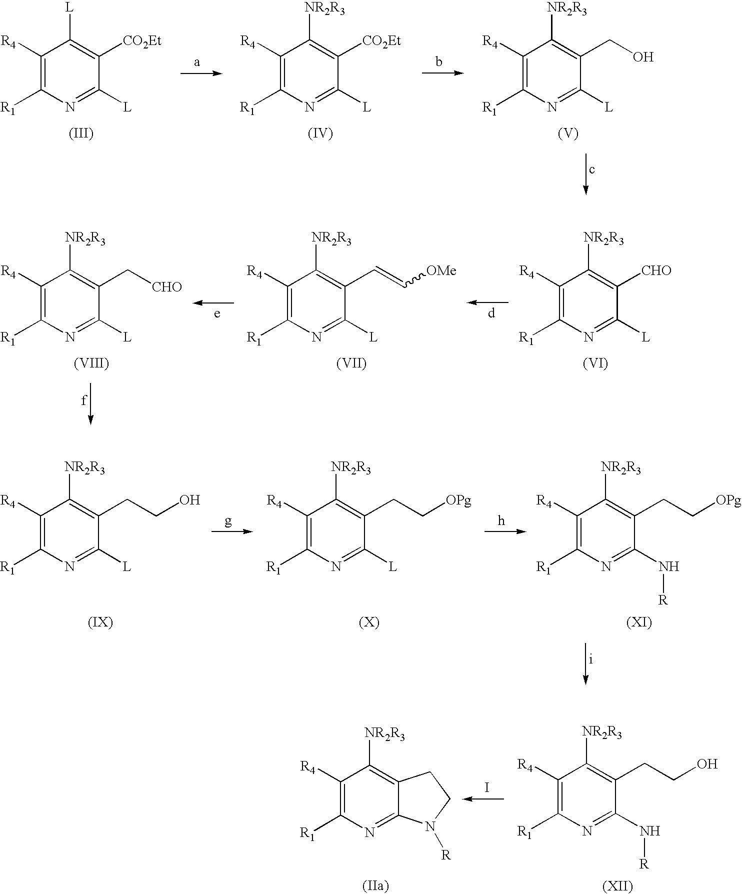
in which
step a stands for conversion of the leaving group L, selected in a group consisting from: halogen or reactive residue of sulphonic acid (e.g. mesylate, tosylate), preferably chloride, in the amino group of compounds (IV), by reaction with the suitable amine NR2R3 in basic conditions;
step b stands for reduction of the ester group with a suitable reducing agent (such as DIBAl-H) to hydroxy group of compounds (V);
step c stands for oxidation of the hydroxy group with a suitable oxidising agent (such as Dess-Martin periodinane) to aldehyde group of compound (VI);
step d stands for formation of the aldehyde group of compounds (VIII) by Wittig reaction in the usual conditions, through formation of enol ether followed by acid hydrolysis (step e);
step f stands for reduction of the aldehyde group with a suitable reducing agent (such as NaBH4) to hydroxy group of compounds (IX);
step g stands for conversion of the hydroxy group in the suitable protecting group of compounds (X)(such as TBS: tert-butyldimethylsilyl);
step h stands for Buchwald reaction by coupling with the suitable amine RNH2;
step i stands for deprotection reaction to give the hydroxy group of compounds (XII);
step l stands for intramolecular cyclisation by heating after conversion of the hydroxy group of compounds (XII) in a suitable leaving group (such as bromide, by reaction with CBr4 and PPh3) to give the final compounds (IIa).

16-18. (canceled)

19. A compound according to claim 1, for use in the treatment of conditions mediated by CRF (corticotropin-releasing factor).

20. A pharmaceutical composition comprising a compound of claim 1, in admixture with one or more physiologically acceptable carriers or excipients.

21. A method for the treatment of a mammal, including man, in particular in the treatment of conditions mediated by CRF (corticotropin-releasing factor), comprising administration of an effective amount of a compound of claim 1.

22. A method of treating depression and anxiety comprising administering a safe and effective amount of a compound according to claim 1 to a patient in need thereof.

23. A method of treating IBS (irritable bowel disease) and IBD (inflammatory bowel disease) comprising administering a safe and effective amount of a compound according to claim 1 to a patient in need thereof.
